SEQUENCE LISTING (9 sequences)

SEQ ID NO: 1 - DNA, 17 bp, Artificial Sequence
  TMPRSS2-ERG QRTPCR Sense Primer
cgcgagctaagcaggag

SEQ ID NO: 2 - DNA, 20 bp, Artificial Sequence
  TMPRSS2-ERG QRTPCR Anti-Sense Primer
cgactggtcctcactcacaa

SEQ ID NO: 3 - DNA, 21 bp, Artificial Sequence
  TMPRSS2-ERG QRTPCR Probe
cgcggcaggaagccttatcag

SEQ ID NO: 4 - DNA, 25 bp, Artificial Sequence
  PSA ARE ChIP Sense Primer
gcctggatctgagagagatatcatc

SEQ ID NO: 5 - DNA, 25 bp, Artificial Sequence
  PSA ARE ChIP Anti-Sense Primer
acacctttttttttctggattgttg

SEQ ID NO: 6 - DNA, 25 bp, Artificial Sequence
  TMPRSS2 ARE ChIP Sense Primer
tggtcctggatgataaaaaaagttt

SEQ ID NO: 7 - DNA, 20 bp, Artificial Sequence
  TMPRSS2 ARE ChIP Anti-Sense Primer
gacatacgccccacaacaga

SEQ ID NO: 8 - DNA, 19 bp, Artificial Sequence
  siRNA sequence complementary to the luciferase gene
cgtacgcggaatacttcga

SEQ ID NO: 9 - DNA, 21 bp, Artificial Sequence
  siRNA sequence complementary to the HDAC6 gene
ctgcaagggatggatctgaac

(19) United States
(12) Patent Application Publication
(10) Pub. No.: US 2011/0195432 A1
(43) Pub. Date: Aug. 11, 2011

(54) METHODS FOR THE SELECTION OF THERAPEUTIC TREATMENTS FOR CANCER

(76) Inventor: Joshi Alumkal, Portland, OR (US)

(21) Appl. No.: 13/123,236

(22) PCT Filed: Oct. 13, 2009
(86) PCT No.: PCT/US09/60493

§ 371 Date: Apr. 7, 2011

Related U.S. Application Data
(60) Provisional application No. 61/105,375, filed on Oct. 14, 2008.

(51) Int. Cl. G01N 33/573; G01N 21/64; G01N 21/76; C12Q 1/34

(52) U.S. Cl. 435/7.4; 435/18

(57) ABSTRACT

Methods are disclosed for identifying subjects that would benefit from treatment with an inhibitor of HDAC6 deacetylase activity. The methods can include contacting a sample obtained from the subject with an of HDAC6 deacetylase activity detecting the amount of HDAC6 protein present in the tumor sample in comparison with a control. Such methods can be used to determine if a tumor in a subject is sensitive to treatment with an inhibitor of HDAC6 deacetylase activity. Also disclosed are methods for monitoring a response to a cancer treatment. Such methods are particularly useful in selecting a compound or multiple compounds for the treatment of cancer in a subject. Methods are also disclosed for identifying agents that inhibit cancer. The methods can include contacting at least one cell with a test agent and detecting an amount of HDAC6 protein in the cell in comparison to a control.

### CROSS-REFERENCE TO RELATED APPLICATIONS

This application claims priority to U.S. Provisional Application No. 61/105,375 filed Oc. 14, 2008, herein incorporated by reference.

### ACKNOWLEDGEMENT OF GOVERNMENT SUPPORT

Work in this application was funded by the Flight Attendant Medical Research Institute and National Institutes of Health award number 1KL2 RR024141 01 through the Oregon Clinical and Translational Research Institute (OCTRI), grant number UL1 RR024140 from the National Center for Research Resources (NCRR), a component of the National Institutes of Health (NIH), and NIH Roadmap for Medical Research. The United States Government has certain rights in this invention.

### FIELD

This application relates to the field of cancer, and specifically to methods of determining the efficacy of treatments for cancer.

### BACKGROUND

One of every three cancers diagnosed in American males is of prostatic origin, making prostate cancer the most commonly diagnosed malignancy in males in the United States (Berges et al. Clin. Cancer Res. 1:473-480, 1995). The incidence of prostate cancer in the U.S. has not been decreased by changes in lifestyle; in fact, the incidence rate of clinical prostate cancer has increased steadily since the 1930's (Pinski et al. Cancer Res. 61:6372-6, 2001). Prostate cancer incidence increases with age more rapidly than any other type of cancer; less than 1% of prostate cancers are diagnosed in men less than 50 years of age (Furuya et al. Cancer Res. 54:6167-75, 1994). Thus, as the life expectancy of the male population increases over time, the incidence of clinical prostate cancer will also increase (Furuya et al. Cancer Res. 54:6167-75, 1994).

Present treatment for prostate cancer includes radical prostatectomy, radiation therapy, or hormonal therapy. With surgical intervention, complete eradication of the tumor is not always achieved and the observed re-occurrence of the cancer (12-68%) is dependent upon the initial clinical tumor stage (Zietman et al., Cancer 71:959, 1993). Thus, alternative methods of treatment including prophylaxis or prevention are desirable.

Medical castration with oral estrogen (androgen ablation) was the first effective systemic therapy for cancer, and is still used today. However, oral estrogen therapy is being supplanted by other forms of androgen deprivation therapy (ADT), such as administration of luteinizing hormone-releasing hormone (LHRH) analogs, (Lupron®, Viadur®, Eligard®, Zoladex® and Trelstar®), LHRH antagonists, such as Plenaxis®), and anti-androgens such a Eulexin®, Casodex®, and Nilandron®.

The oncologic benefits of ADT may be partially offset, however, by a reduction in quality of life due to adverse effects. In addition to the well-recognized adverse consequences of ADT, recent evidence suggests that ADT is associated with dyslipidemia, impaired glucose metabolism, adverse body compositional changes, and osteoporosis. Thus, there is a pressing need to develop less toxic forms of ADT.

Alternative approaches to the treatment of prostate cancer have been proposed. For example, high consumption of broccoli and other cruciferous vegetables is associated with a lower risk of prostate cancer development, and sulforaphane, a component of broccoli, has been shown to be an effective chemopreventive and anti-cancer agent in multiple pre-clinical systems. However the molecular mechanisms that underlie the effects of agents such as sulforaphane on carcinogenesis have not been clarified. In addition, other than measuring tumor reduction in response to sulforaphane treatment, there are not sufficient means for determining whether a subject is responding to sulforaphane treatment, for example to determine if a subject should be continued on a sulforaphane treatment regime.

### SUMMARY

It is shown herein that sulforaphane enhances HSP90 acetylation, thereby inhibiting its association with AR. Moreover, AR is subsequently degraded in the proteasome, which leads to reduced AR target gene expression and reduced AR occupancy at its target genes. Sulforaphane also inhibits HDAC6 deacetylase activity (and thus increases the acetylation of alpha-tubulin), and the effects of sulforaphane on AR protein are abrogated by over-expression of HDAC6 and mimicked by HDAC6 siRNA. Based on the observation that inactivation by sulforaphane of HDAC6-mediated HSP90 deacetylation and consequent attenuation of AR signaling, methods are provided that are useful in anti-cancer therapy, such as an anti-prostate cancer therapy.

Methods are disclosed for identifying subjects that would benefit from treatment with an inhibitor of HDAC6 deacetylase activity, such as sulforaphane. In some embodiments, the methods include contacting a sample obtained from a subject with an inhibitor of HDAC6 deacetylase activity. The amount of HDAC6 protein or HDAC6 activity present in the sample is detected and compared with a control. In some examples, one or more of the following HDAC6 activities are detected (for example in combination with detecting HDAC6 protein): levels of alpha-tubulin acetylation, HSP90 acetylation, androgen receptor (AR) protein, prostate specific antigen (PSA) protein, and/or ERG protein (such as the TMPRSS2-ERG fusion protein). A reduction in the amount of HDAC6 in the sample relative to the control indicates that the subject is a candidate for treatment with an inhibitor of HDAC6 deacetylase activity. Similarly, detection of the following HDAC6 activities, such as an increase in the levels of acetylated alpha-tubulin or acetylated HSP90, or a decrease in the levels of AR, PSA and/or ERG relative to a control indicates that the subject is a candidate for treatment with an inhibitor of HDAC6 deacetylase activity. In some examples, the biological sample is a tumor sample and the methods can be used to determine if a tumor in a subject is sensitive to treatment with an inhibitor of HDAC6 deacetylase activity.

Also disclosed herein are methods for monitoring a response to a cancer treatment. In some embodiments, these methods include detecting an amount of HDAC6 protein or HDAC6 activity present in a sample obtained from a subject that is undergoing a cancer treatment. In some examples, one or more of the following HDAC6 activities are detected (for example in combination with detecting HDAC6 protein): levels of alpha-tubulin acetylation, HSP90 acetylation, AR protein, PSA protein, and/or ERG protein (such as the TMPRSS2-ERG fusion protein). In some examples the sample is a tumor sample. The amount of HDAC6 protein present in the sample is detected and compared with a control. A decrease in the amount of HDAC6 protein or activity relative to the control indicates that the subject is responding to the cancer treatment. In addition, detection of an increase in the levels of acetylated alpha-tubulin or acetylated HSP90, or a decrease in the levels of AR, PSA and/or ERG relative to a control indicates that the subject is responding to the cancer treatment. Conversely an increase or maintenance in the amount of HDAC6 protein or activity relative to the control can indicate that the subject is not responding to the cancer treatment and a different treatment should be selected. For example, detection of a decrease in the levels of acetylated alpha-tubulin or acetylated HSP90, or an increase in the levels of AR, PSA and/or ERG relative to a control indicates that the subject is not responding to the cancer treatment and a different treatment should be selected. Such methods are particularly useful in selecting a compound or multiple compounds for the treatment of cancer in a subject, such as prostate cancer.

Methods are also disclosed for identifying agents that inhibit cancer. In some embodiments the methods include contacting at least one cell with a test agent and detecting an amount of HDAC6 protein (or activity) in the cell. The amount of HDAC6 protein or activity present in the cell is then compared to a control. A reduction in the amount of HDAC6 protein relative to a control indicates that the agent is of use in inhibiting cancer, such as prostate cancer. In some examples, one or more of the following HDAC6 activities are detected (for example in combination with detecting HDAC6 protein): levels of alpha-tubulin acetylation, HSP90 acetylation, AR protein, PSA protein, and/or ERG protein (such as the TMPRSS2-ERG fusion protein), wherein an increase in the levels of acetylated alpha-tubulin or acetylated HSP90, or a decrease in the levels of AR, PSA and/or ERG relative to a control indicates that the agent is of use in inhibiting cancer, such as prostate cancer.

The foregoing and other objects and features of the disclosure will become more apparent from the following detailed description, which proceeds with reference to the accompanying figures.

### BRIEF DESCRIPTION OF THE DRAWINGS

FIGS. 1A and 1B are digital images of sets of Westen blots showing that sulforaphane treatment of prostate cancer cells leads to increased heat shock protein 90 (HSP90) acetyation and dissociation from androgen receptor (AR). FIGS. 1A (LNCaP cells) and 1B (VCaP cells) are digital images of Westen blots for HSP90, showing the results of an immunoprecipitation that was carried out with an anti-acetyl lysine antibody or IgG after treatment with sulforaphane or vehicle for (A) four or (B) six hours. Input protein lysates were probed with antibodies for histone deacetylase 6 (HDAC6), HSP90, acetylated alpha-tubulin, and tubulin and quantified. FIG. 1C is a set of digital images of Westen blots, showing the results of an immunoprecipitation carried out with antibodies to HSP90, AR, acetylated lysine, and IgG after treatment with sulforaphane or vehicle for four hours followed by a Western blot for AR. Input protein lysates were probed with antibodies for AR, HSP90, acetylated tubulin, and tubulin and quantified.

FIGS. 2A and 2B are digital images of Western blots showing that sulforaphane treatment of prostate cancer cells lowers AR protein levels. FIG. 2A is a digital image of a Western blot of protein lysates for AR expression from LNCaP cells treated with increasing doses of sulforaphane, vehicle or TSA (trichostatin A, a pan-HDAC inhibitor) at the indicated time points. GAPDH was used as a loading control. FIG. 2B is a digital image of a Western blot of protein lysates for AR expression from VCaP cells treated with vehicle, increasing doses of sulforaphane, or TSA at 24 hours. GAPDH was used as a loading control. AR and GAPDH levels by Western blot were quantified.

FIGS. 3A and 3B are digital images of Western blots showing that CDDO-Imidazole recapitulates sulforaphane's effects. (FIG. 3A) LNCaP and (FIG. 3B) VCaP cells were treated with vehicle or increasing concentrations of CDDO-Imidazole for 24 hours. Protein lysates were probed with the antibodies indicated. AR, acetylated tubulin, HDAC6, and actin levels by Western blot were quantified.

FIGS. 4A and 4B are are a set of bar graphs showing that high-dose sulforaphane treatment of prostate cancer cells lowers AR transcript levels. (FIG. 4A) Real-time PCR of AR gene expression with cDNA from LNCaP cells treated with vehicle, increasing doses of sulforaphane, or TSA. (FIG. 4B) Real-time PCR of AR gene expression with cDNA from VCaP cells treated with vehicle, increasing doses of sulforaphane, or TSA at 24 hours. The vehicle-treated sample was set to 1. 18S was used as an endogenous control in all assays.

FIGS. 5A, 5B, and 5C are bar graphs showing sulforaphane treatment of prostate cancer cells reduces AR target gene expression. (FIG. 5A) Real-time PCR of PSA expression from LNCaP cells treated with vehicle, increasing doses of sulforaphane, or TSA at the indicated time points. Real-time PCR of PSA (FIG. 5B) and TMPRSS2-ERG (FIG. 5C) gene expression from VCaP cells at 24 hours. The vehicle-treated sample was set to 1. 18S was used as an endogenous control in all assays.

FIGS. 6A-6D are digital images of agarose gels and Western blots showing that sulforaphane treatment of prostate cancer cells depletes AR from androgen response elements (AREs) and lowers ERG protein. (FIG. 6A) LNCaP or (FIG. 6B, 6C) VCaP cancer cells were treated with vehicle or 20 μM sulforaphane for 24 hours. ChIP analysis was used to determine AR occupancy at the indicated gene AREs. Enrichment was calculated compared to the respective input control. (FIG. 6D) Western blot for AR and ERG protein expression from VCaP whole cell lysates treated in parallel with samples from B and C. AR, ERG, and Actin levels by Western blot were quantified.

FIG. 7 is a set of digital images of Western blots showing that proteasome inhibitor (MG132) treatment of LNCaP cancer cells rescues AR protein from sulforaphane treatment. LNCaP cancer cells were treated for 24 hours with sulforaphane 20 μM with or without 10 μM MG132, 300 nM TSA with or without 10 μM MG132, vehicle, or 10 μM MG132. AR levels were determined by Western blot. GAPDH was used as a loading control.

FIG. 8 is digital image of a set of Western blots showing sulforaphane treatment inhibiting HDAC6. Tubulin dimers were either incubated without recombinant HDAC6 or with recombinant HDAC6 in the presence of vehicle, 7.5 or 15 μM sulforaphane, or 400 nM TSA. Levels of HDAC6, acetylated tubulin, and tubulin by Western blot were quantified.

FIGS. 9A and 9B are digital images of sets of Western blots showing ectopic over-expression of HDAC6 attenuates sulforaphane-mediated AR and HDAC6 protein depletion, while HDAC6 siRNA recapitulates the findings observed with sulforaphane. FIG. 9A is a digital image of a Western blot from LNCaP cells that were transfected with pCDNA3.1 or FLAG-HDAC6. 48 hours later, cells were then treated with either vehicle or 15 μM sulforaphane for 16 hours. Levels of FLAG, HDAC6, AR, acetylated alpha-tubulin, and actin were measured by Western blot. FIG. 9B is a digital image of a Western blot from LNCaP cells that were transfected with 100 nM siRNA to either the luciferase gene (si LUC) or HDAC6 gene (si HDAC6). Levels of HDAC6, AR, acetylated alpha-tubulin, and actin were measured by Western blot in protein lysates harvested at the indicated time points. Bands from the HDAC6 siRNA samples were compared to the luciferase control samples from the same time point.

FIGS. 10A and B are bar graphs showing that sulforaphane treatment of prostate cancer cells lowers HDAC6 transcript levels while HDAC6 over-expression does not increase AR transcript levels. LNCaP cells were transfected with pCDNA3.1 or FLAG-HDAC6. Cells were then treated with either vehicle or 15μM sulforaphane. (FIG. 10A) Real-time PCR of HDAC6 expression. (FIG. 10B) Real-time PCR of AR expression. The vehicle-treated sample was set to 1. 18S was used as an endogenous control in all assays.

FIG. 11 is a digital image of a Western blot showing that proteasome inhibitor treatment of prostate cancer cells rescues HDAC6 protein from sulforaphane treatment. LNCaP cancer cells were treated for 16 hours with vehicle without (lane 1) or with 20 μM MG132 (lane 2), 20 μM sulforaphane without (lane 3) or with 20 μM MG132 (lane 4), or 300 nM TSA without (lane 5) or with 20 μM MG132 (lane 6). HDAC6 and Actin levels by Western blot were quantified.

FIG. 12 is a schematic representation of sulforaphane attenuation of AR signalling via HDAC6 inactivation. Normally, HSP90 is deacetylated by HDAC6, which enables it to chaperone client proteins such as AR. HDAC6 also deacetylates alpha-tubulin. With sulforaphane treatment, HDAC6 is inhibited or targeted for protein degradation. This leads to hyperacetylated alpha-tubulin. The HDAC6 inactivation with sulforaphane also leads to hyperacetylated, inactive HSP90 protein, which dissociates from AR, and AR is then targeted for protein degradation. Consequently, AR binding to its target gene androgen response elements (ARE), including TMPRSS2-ERG, is diminished, which reduces their expression.

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. 1.822. In at least some cases, only one strand of each nucleic acid sequence is shown, but the complementary strand is understood as included by any reference to the displayed strand.

SEQ ID NOS: 1-2 are primers used to amplify the TMPRSS2-ERG fusion.

SEQ ID NO: 3 is a probe used to detect the TMPRSS2-ERG fusion.

SEQ ID NOS: 4-7 are ChIP PCR primer sequences.

SEQ ID NO: 8 is a luciferase siRNA sequence.

SEQ ID NO: 9 is an HDAC6 siRNA sequence.

### DETAILED DESCRIPTION

I. Summary of Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

The singular forms “a,” “an,” and “the,” refer to both the singular as well as plural, unless the context clearly indicates otherwise, thus a cell can mean either one cell or multiple cells. Similarly, the word “or” is intended to include “and” unless the context clearly indicates otherwise. As used herein, the term “comprises” means “includes.” Hence comprising A or B″ means A, B, or A and B. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the disclosure, the following explanations of terms are provided:

Administration: The introduction of a composition into a subject by a chosen route. For example, if the chosen route is intravenous, the composition is administered by introducing the composition into a vein of the subject. In some examples an inhibitor of HDAC6 deacetylase activity, such as sulforaphane, is administered to a subject.

Alpha-tubulin: Dimers of α-tubulin and β-tubulin form microtubules. Each has a molecular weight of approximately 55 kD. Subtypes of alpha-tubulin include TUBA1A, TUBA1B, TUBA1C, TUBA3C, TUBA3D, TUBA3E, TUBA4A, and TUBA8.

Exemplary alpha-tubulin nucleic acid and amino acid sequences can be found on GENBANK®, for example at Accession Nos. CAA30026.1 and X06956.1 (Homo sapiens); AAA42306.1 and AH002269.1 (Rattus norvegicus); and AAA40507.1 and NM_{—}011653.2 (Mus musculus) as available Oct. 13, 2009, are incorporated herein by reference in their entirety.

Animal: Living multi-cellular vertebrate organisms, a category that includes, for example, mammals and birds. The term mammal includes both human and non-human mammals. Similarly, the term “subject” includes both human and veterinary subjects. For example, administration to a subject can include administration to a human subject or a veterinary subject, such as a mouse.

Antibody: A polypeptide ligand comprising at least a light chain or heavy chain immunoglobulin variable region which specifically recognizes and binds an epitope of an antigen or a fragment thereof, for example an epitope on HDAC6. Antibodies can be composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

The term antibody includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds an antigen of interest has a specific V_{H} region and the V_{L} region sequence, and thus specific CDR sequences. Antibodies with different specificities (due to different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected, or a progeny thereof. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Androgen Receptor (AR): A nuclear receptor activated by binding of either of the androgenic hormones testosterone or dihydrotestosterone. Dysregulation of the androgen receptor (also known as nuclear receptor subfamily 3, group C, member 4 or NR3C4) is believed to participate in prostate cancer, resulting in an abnormal profile of AR-regulated genes, which include cell cycle regulators, transcription factors, and those proteins important for cell survival, lipogenesis, and secretion.

Exemplary AR nucleic acid and amino acid sequences can be found on GENBANK®, for example at Accession Nos. NM_{—}000044.2 and NP_{—}000035 NP_{—}001011645 (Homo sapiens); NM_{—}001003053.1 and NP_{—}001003053 (Canis familiaris); NM_{—}012502.1 and NP_{—}036634 (Rattus norvegicus); and NM_{—}013476.3 and NP_{—}038504 (Mus musculus) as available Oct. 8, 2008, are incorporated herein by reference in their entirety.

Binding affinity: Affinity of an antibody for an antigen, such as HDAC6. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸ M, at least about 2.0×10⁻⁸M, at least about 2.5×10⁻⁸M, at least about 3.0×10⁻⁸M, at least about 3.5×10⁻⁸M, at least about 4.0×10⁻⁸M, at least about 4.5×10⁻⁸M, or at least about 5.0×10⁻⁸ M.

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating prostate cancer, such as an anti-neoplastic agent. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993. Combination chemotherapy is the administration of more than one agent to treat cancer, for example to treat prostate cancer. In some embodiments an inhibitor of HDAC6 deacetylase activity (such as sulforaphane) is a chemotherapeutic agent. Some common chemotherapy drugs used to treat cancer, such as prostate cancer treatment include mitozantrone, doxorubicin, vinblastine, paclitaxel, docetaxel, estramustine phosphate and etoposide.

Contacting: “Contacting” includes in solution and solid phase, for example contacting a sample with a test agent or an inhibitor of HDAC6 deacetylase activity. The test agent may also be a combinatorial library for screening a plurality of compounds. In another example, contacting includes contacting a sample with an antibody, for example contacting a sample that contains or is suspected of containing HDAC6, with an antibody that specifically binds HDAC6.

Control: A reference standard. A control can be a known value indicative of basal levels or amounts of HDAC6 protein. A control can also be a cellular control, for example a cell not contacted with a test agent or inhibitor of HDAC6 deacetylase activity a cell contacted with vehicle or carrier alone, a non-cancerous cell, a cell obtained from the subject from an earlier time point, or any combination thereof. A difference between a test sample and a control can be an increase or conversely a decrease. The difference can be a qualitative difference or a quantitative difference, for example a statistically significant difference. In some examples, a difference is an increase or decrease in amount, relative to a control, of at least about 1%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

Detect: To determine if an agent (such as a signal or particular nucleotide nucleic acid probe, amino acid, or protein, for example HDAC6 protein) is present or absent. In some examples, this can further include quantification, for example the quantification of the amount of HDAC6 in a sample.

Diagnostic: Identifying the presence or nature of a pathologic condition, such as, but not limited to cancer, such as prostate cancer. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (for example severity) of a pathologic condition, such as cancer (for example prostate cancer), or metastasis.

Effective amount or Therapeutically effective amount: The amount of agent, such as an inhibitor of HDAC6 activity, that is an amount sufficient to prevent, treat (including prophylaxis), reduce and/or ameliorate the symptoms and/or underlying causes of any of a disorder or disease, for example to prevent, inhibit, and/or treat prostate cancer. In some embodiments, an “effective amount” is sufficient to reduce or eliminate a symptom of a disease, such as prostate cancer. An amount sufficient to achieve a desired biological effect, for example an amount that is effective to decrease the size (e.g., volume), side effects and/or metastasis of prostate cancer. In one example, it is an amount sufficient to decrease the symptoms or effects of a prostate carcinoma, such as the size of the tumor. In particular examples, it is an amount effective to decrease the size of a prostate tumor and/or prostate metastasis by at least 30%, 40%, 50%, 70%, 80%, 90%, 95%, 99% or even 100% (complete elimination of the tumor). In some examples an effective amount is the amount affective to produce the desired effect in vitro, for example the amount of an inhibitor of HDAC6 deacetylase activity needed to reduce the amount of HDAC6 protein in a cell.

Heat shock protein 90 (HSP90): Heat shock proteins protect cells when stressed by elevated temperatures. Hsp90 is one of the most common of the heat related proteins, and is about 90 kiloDaltons. HSP90 is deacetylated by HDAC6 HSP90, leading to activation of HSP90, enhanced binding of HSP90 to client proteins including the androgen receptor (AR) protein, and consequently attenuated degradation of client proteins including AR. In contrast, exposure of HSP90 to an HDAC6 inhibitor increases acetylation of HSP90, and thus increased degradation of AR protein.

Exemplary HSP90 nucleic acid and amino acid sequences can be found on GENBANK®, for example at Accession Nos. AAI08696.1, AAH09195.1, BC009195.2, and BC108695.1 (Homo sapiens); NP_{—}001073105.1 and NM_{—}001079637.1 (Bos Taurus); AAB23369.1 and 545392.1 (Rattus norvegicus); and CAX15714.1 and AL596265.12 (Mus musculus) as available Oct. 13, 2009, are incorporated herein by reference in their entirety.

Histone deacetylase 6 (HDAC6): One of the members of the histone deacetylase family. HDAC6 belongs to class II of the histone deacetylase/acuc/apha family and possesses histone deacetylase activity. HDAC6 is a cytoplasmic non-histone protein deacetylase whose substrates include alpha-tubulin and the HSP90 chaperone protein. When HDAC6 deacetylates HSP90, this leads to activation of HSP90, enhanced binding of HSP90 to client proteins including the androgen receptor (AR) protein, and consequently attenuated degradation of client proteins including AR. HDAC6 activity refers to the direct or indirect biological effects or actions of the HDAC6 protein, such as effects on deacetylation of alpha-tubulin and HSP90, and effects on increasing the levels of AR, PSA, and ERG (such as TMPRSS2-ERG) proteins.

Exemplary HDAC6 nucleic acid and amino acid sequences can be found on GENBANK®, for example at Accession Nos. NM_{—}006044 and NP_{—}006035 (Homo sapiens); XM_{—}850269.1 and XP_{—}855362.1 (Canis familiaris); XM_{—}591306.3 and XP_{—}591306.3 (Bos Taurus); XM_{—}228753.4 and XP_{—}228753.4 (Rattus norvegicus); and NM_{—}010413.21 and NP_{—}034543.21 (Mus musculus) as available Oct. 8, 2008, are incorporated herein by reference in their entirety.

Inhibiting or treating a disease: Inhibiting the full development of a disease or condition, for example, for example cancer, such as prostate cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, for example metastatic prostate cancer.

Inhibit: To reduce to a measurable extent. For example to reduce enzymatic activity, such as HDAC6 histone deacetylase activity. An inhibitor is a compound that inhibits enzymatic activity. For example an agent, such as sulforaphane, that inhibits the enzymatic activity of HDAC6, is an HDAC6 inhibitor. For example, such agents can do one or more of the following increase acetylation of alpha-tubulin, increase acetylation of HSP90, decrease levels of AR, decrease levels of ERG (such as TMPRSS2-ERG), or decrease levels of PSA. Examples of HDAC6 inhibitors that can be used in the disclosed methods are sulforaphane, tubacin, belinostat (PXD101), tau protein (see Perez et al., J. Neurochem. 109(6):1756-66, 2009), as well as the pyridylalanine-containing hydroxamic acids disclosed in Schafer et al. (Chem Med Chem. 4(2):283-90, 2009, herein incorporated by reference) among others.

Immunoassay: A biochemical test that measures the presence or concentration of a substance in a sample, such as a biological sample, using the reaction of an antibody to its cognate antigen, for example the specific binding of an antibody to a protein, such as HDAC6. Both the presence of antigen or the amount of antigen present can be measured. In some examples, the amount of HDAC6 protein is measured and HDAC6 is the antigen and the presence and amount of HDAC6 is determined or measured.

Measuring the quantity of antigen (such as HDAC6, AR, PSA, ERG, acetylated alpha-tubulin or acetylated HSP90) can be achieved by a variety of methods. One of the most common is to label either the antigen or antibody with a detectable label. Specific, non-limiting examples of labels include fluorescent tags, enzymatic linkages, and radioactive isotopes (for example ¹⁴C, ³²P, ¹²⁵I, and ³H isotopes and the like). In some examples an antibody that specifically binds HDAC6 is labeled. Methods for labeling and guidance in the choice of labels appropriate for various purposes are discussed for example in Sambrook et al. (Molecular Cloning: A Laboratory Manual, Cold Spring Harbor, New York, 1989) Ausubel et al. (In Current Protocols in Molecular Biology, John Wiley & Sons, New York, 1998), and Harlow & Lane, (Antibodies, A Laboratory Manual, Cold Spring Harbor Publications, New York, 1988)),

Label: A detectable compound or composition, which can be conjugated directly or indirectly to another molecule, such as an antibody (for example an antibody that specifically binds HDAC6 or other antigen provided herein) or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides (such as ³⁵S or ¹³¹I), fluorescent labels (such as fluoroscein istothiocyanate (FITC), rhodamine, lanthanide phosphors, cyanine dyes, fluorescent proteins, such as GFP), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

Mass spectrometry: A method wherein a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography. In some examples mass spectrometry is used to measure the amount of HDAC6 (or AR, PSA, ERG, acetylated alpha-tubulin or acetylated HSP90) present in a sample, such as a sample obtained from a subject.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. In some examples, a polypeptide is an HDAC6, AR, PSA, ERG, acetylated alpha-tubulin or acetylated HSP90, polypeptide.

Pharmaceutically acceptable carriers: The pharmaceutically acceptable carriers useful in conjunction with test agents and inhibitors of HDAC6 deacetylase activity are conventional. Remington's Pharmaceutical Sciences, by E. W. Martin, Mack Publishing Co., Easton, Pa., 15th Edition (1975), describes compositions and formulations suitable for pharmaceutical delivery a therapeutic agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical or therapeutic agent: A chemical compound or a composition capable of inducing a desired therapeutic or prophylactic effect when properly administered to a subject or a cell. A pharmaceutical agent can be an inhibitor of HDAC6 histone deacetylase activity, for example sulforaphane.

Prognosis: The probable course or outcome of a disease process. In several examples, the prognosis of a subject with cancer can indicate the likelihood of survival and/or the likelihood of metastasis. The prognosis of a subject with cancer can indicate the likelihood that the subject will survive for a period of time, such as about one, about two, about three, about four, about five or about ten years. The prognosis of a subject with cancer can also indicate the likelihood of a cure, of the likelihood that the subject will remain disease-free following treatment for a period of time, such as about one, about two, about three, about four, about five or about ten years.

Prostate Cancer: A malignant tumor, generally of glandular origin, of the prostate. Prostate cancers include adenocarcinomas and small cell carcinomas. Many prostate cancers express prostate specific antigen (PSA).

Prostate Specific Antigen (PSA): A glycoprotein manufactured almost exclusively by the prostate, and also known as kallikrein III, seminin, semenogelase, γ-seminoprotein and P-30 antigen. PSA is a serine protease, produced by normal prostatic tissue, and secreted exclusively by the epithelial cells lining prostatic acini and ducts. Prostate specific antigen can be detected at low levels in the sera of healthy males without clinical evidence of prostate cancer. However, during neoplastic states, circulating levels of this antigen increase dramatically, correlating with the clinical stage of the disease. Prostate specific antigen is used as a marker for prostate cancer.

Exemplary PSA nucleic acid and amino acid sequences can be found on GENBANK®, for example at Accession Nos. AAA58802 and M27274.1 (Homo sapiens); and AAZ82258 and X73560.1 (Macaca mulatta) as available Oct. 8, 2008, are incorporated herein by reference in their entirety.

Sample: A biological sample obtained from a subject, such as a human or other primate or mammal, which contains for example nucleic acids and/or proteins. As used herein, biological samples include all clinical samples useful for detection of HDAC6 (and in some examples also AR, PSA, ERG, acetylated alpha-tubulin, and acetylated HSP9) in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In particular embodiments, the biological sample is obtained from a subject, such as in the form of a tumor sample, for example a prostate tumor sample.

Specific binding agent: An agent that binds substantially only to a defined target. In some embodiments, a specific binding agent is an antibody that specifically binds HDAC6, AR, PSA, ERG, acetylated alpha-tubulin, acetylated HSP90 or functional fragment thereof.

The term “specifically binds” refers to the preferential association of an antibody or other ligand, in whole or part, with a cell bearing that antigen and not to cells or tissues lacking that antigen. It is, of course, recognized that a certain degree of non-specific interaction may occur between a molecule and a non-target cell. Nevertheless, specific binding may be distinguished as mediated through specific recognition of the antigen. Although selectively reactive antibodies bind antigen, they may do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a cell or tissue expressing the target epitope as compared to a cell or tissue lacking this epitope. A variety of immunoassay formats are appropriate for selecting antibodies or other ligands specifically immunoreactive with a particular protein. For example, solid-phase

ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Sulforaphane: A compound ((R)-1-isothiocyanto-4-methyl-sulfonyl butane or 1-Isothiocyanato-4-methylsulfinylbutane) that can be found in cruciferous vegetables such as brussel sprouts, broccoli, cabbage, cauliflower, bok choy, kale, collards, broccoli sprouts, chinese broccoli, broccoli raab, kohlrabi, mustard, turnip, radish, rocket, and watercress. Sulforaphane is a derivative of glucoraphanin and is shown herein to be an inhibitor of histone deacetylase 6 (HDAC6) activity, thereby suppressing the stability and function of the AR.

Transmembrane protease, serine 2-v-ets erythroblastosis virus E26 oncogene homolog (avian) (TMPRSS2-ERG): The TMPRSS2-ERG fusion is observed in around 90% of tumors that overexpress the oncogene ERG. Translocation of TMPRSS2 to the ERG gene, found in a high proportion of human prostate cancer, results in overexpression of the 3′-ERG sequences joined to the 5′-TMPRSS2 promoter. When is it stated herein that TMPRSS2-ERG or ERG is detected, in some examples this includes detection of the TMPRSS2-ERG fusion protein. In some examples, it also includes detection of ERG.

Exemplary TMPRSS2-ERG nucleic acid and amino acid sequences can be found on GENBANK®, for example at Accession Nos. ACA81385.1 and EU432099.1. Exemplary ERG nucleic acid and amino acid sequences can be found on GENBANK® for example at Accession Nos. NP_{—}005229.1, NP_{—}001137292.1, NP_{—}005230.1 and NM_{—}005238.3 as available Oct. 13, 2009, are incorporated herein by reference in their entirety.

Tumor or cancer: The product of neoplasia is a neoplasm (a tumor or cancer), which is an abnormal growth of tissue that results from excessive cell division. A tumor that does not metastasize is referred to as “benign.” A tumor that invades the surrounding tissue and/or can metastasize is referred to as “malignant.” Neoplasia is one example of a proliferative disorder.

Examples of hematological cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia.

Examples of solid cancers, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). A specific non-limiting example of cancer is prostate cancer.

Tissue: A plurality of functionally related cells. A tissue can be a suspension, a semi-solid, or solid. Tissue includes cells collected from a subject such as blood, cervix, uterus, lymph nodes breast, skin, and other organs. In some examples tissue is a sample of prostate tumor cells.

### II. Overview of Several Embodiments

Prostate cancer remains a common and sometimes lethal cancer. The vast majority of men who die from prostate cancer have castrate-resistant prostate cancers at the time of their death, in which androgen receptor (AR) is still active. It is clear that AR protein is an important target in all phases of this disease.

Sulforaphane is an isothiocyanate compound naturally found in broccoli and other cruciferous vegetables. Isolated sulforaphane is known to prevent and induce regression of prostate and other malignancies in pre-clinical models, but the mechanisms by which it exerts these effects remain unclear. As disclosed herein, sulforaphane treatment suppresses growth of prostate cancer cells by inhibiting HDAC6-mediated protein deacetylation of HSP90, which chaperones and stabilizes the AR. Sulforaphane treatment leads to AR dissociation from HSP90, AR degradation, and attenuated AR signaling, for example diminishing the expression of down-stream target genes such as TMPRSS2-ERG and PSA. These elements can be measured to determine is a subject is amenable to treatment with sulforaphane or other HDAC6 inhibitor.

What was particularly surprising was that after sulforaphane treatment the levels of HDAC6 protein is reduced in cells contacted with sulforaphane. This result was particularly surprising because one would expect total HDAC6 levels to stay the same or possibly increase to compensate for the inhibition of HDAC6 activity. However, this is not what occurred. Upon sulforaphane treatment, HDAC6 protein levels actually decreased. This reduction in HDAC6 protein levels represents a novel marker for therapeutic efficacy of prostate cancer treatments. Furthermore, because HSP90 has been implicated in stabilizing gene products involved in other cancers, monitoring the HDAC6 protein levels in response to therapeutic treatment of represents a novel, more easily measurable approach to monitoring therapies for tumors besides prostate tumor. For example, HSP90 chaperones ErbB2 (protein product of the Her2Neu gene) in breast cancer, bcr-abl in chronic myelogenous leukemia (CML), and c-kit in gastrointestinal stromal tumors (GIST). Thus, monitoring the amount of HDAC6 protein present in tumor samples obtained from subjects with these cancers can be used to measure efficacy of treatment, for example treatment with an inhibitor of HDAC6 deacetylase activity.

A. Methods for Selection of Subjects for Treatment with HDAC6

Inhibitors

Disclosed herein are methods for identifying a subject that is a candidate for treatment with an inhibitor of HDAC6 deacetylase activity, for example to treat a tumor in the subject, such as a prostate tumor. The method can include contacting a sample, such as a tumor sample, from the subject (such as a mammalian or primate subject) with an effective amount of inhibitor of HDAC6 deacetylase activity and detecting an amount of HDAC6 protein or HDAC6 activity in the sample. It is determined if the amount of HDAC6 protein (or activity) present in the sample is reduced, increased or remains the same relative to a control (such as an amount of HDAC6 protein or activity present in a normal prostate sample, for example a reference value or range of values representing the expected HDAC6 protein levels in a normal prostate cell or sample).

A reduction in the amount of HDAC6 protein or activity in the sample relative to the control (such as an amount of HDAC6 protein present in a normal prostate sample, for example a reference value or range of values representing the expected HDAC6 protein levels in a normal prostate cell or sample) indicates that the subject is a candidate for treatment with an inhibitor of HDAC6 deacetylase activity, such as sulforaphane, tubacin, belinostat, or others known in the art. For example, a reduction of at least 10%, at least 20%, at least 30%, at least 50%, at least 75%, at least 80%, at least 90%, at least 95%, at least 98% or even at least 99%, relative to the control, indicates that the subject (such as a human subject with prostate cancer) is a candidate for treatment with an inhibitor of HDAC6 deacetylase activity.

Conversely, an increase or maintenance in the amount of HDAC6 protein present in the sample relative to a control, such as an amount of HDAC6 protein present in a normal prostate sample (for example a reference value or range of values representing the expected HDAC6 protein levels in a normal prostate cell or sample) would indicate that the subject is not a candidate for treatment with an inhibitor of HDAC6 deacetylase activity. For example, an increase of at least 10%, at least 20%, at least 30%, at least 50%, at least 75%, at least 80%, at least 90%, at least 100%, at least 200% or even at least 500%, relative to the control, indicates that the subject (such as a human subject with prostate cancer) is not a candidate for treatment with an inhibitor of HDAC6 deacetylase activity.

In some examples, one or more of the following HDAC6 activities are measured, for example in combination with measuring or detecting HDAC6 protein, after the sample is contacted with an inhibitor of HDAC6 deacetylase activity (e.g., sulforaphane, tubacin, or belinostat): acetylated HSP90 or alpha-tubulin levels (for example using antibodies specific for the acetylated state, immunoprecipitations, or mass spec), or levels of AR, PSA, or TMPRSS2-ERG protein (for example using antibodies specific for these proteins). An increase in HSP90 or alpha-tubulin acetylation (e.g., an increase in acetyl HSP90 or acetyl alpha-tubulin), or a decrease in levels of AR, PSA, or TMPRSS2-ERG, relative to a control, indicates that the subject is a candidate for treatment with an inhibitor of HDAC6 deacetylase activity, such as sulforaphane, tubacin, or belinostat. For example, an increase in acetyl HSP90 or acetyl alpha-tubulin of at least 10%, at least 20%, at least 30%, at least 50%, at least 75%, at least 80%, at least 90%, at least 95%, at least 98%, at least 99%, at least 200%, at least 300%, or even at least 500%, or a reduction of in AR, PSA, or TMPRSS2-ERG of at least 10%, at least 20%, at least 30%, at least 50%, at least 75%, at least 80%, at least 90%, at least 95%, at least 98% or even at least 99%, relative to the control, indicates that the subject (such as a human subject with prostate cancer) is a candidate for treatment with an inhibitor of HDAC6 deacetylase activity.

The disclosed methods are useful in determining if a tumor in a subject is sensitive to treatment with an inhibitor of HDAC6 deacetylase activity, for example treatment with sulforaphane, tubacin, belinostat, or other HDAC6 inhibitor known in the art. In some examples, the inhibitor of HDAC6 deacetylase activity is sulforaphane and the sample obtained from the subject is contacted with sulforaphane.

In some examples, the sample, such as a tumor sample obtained from the subject, is contacted with an effective amount of an inhibitor of HDAC6 deacetylase activity (such as sulforaphane, tubacin, belinostat, or other inhibitor of HDAC6 deacetylase activity known in the art) at a concentration of about 1 picomolar to about 100 mmolar, such as a concentration of test agent of about 1 picomolar, about 10 picomolar, about 100 picomolar, about 1 nanomolar, about 10 nanomolar, about 100 nanomolar, about 1 micromolar, about 10 micromolar, about 100 micromolar, 1 millimolar, about 10 millimolar, about 100 millimolar, about 1 micromolar, or even about 10 micromolar or greater.

In some examples, the sample, such as a tumor sample obtained from a subject, is contacted with different concentrations of the inhibitor of HDAC6 deacetylase activity (such as sulforaphane or other inhibitor of HDAC6 deacetylase known in the art), for example to determine reduction in the amount of HDAC6 protein or activity present (for example to determine increase in the amount of HSP90 acetylation or alpha-tubulin acetylation (e.g., an increase in acetyl HSP90 or acetyl alpha-tubulin) or to determine reduction in the amount of AR, PSA, or TMPRSS2-ERG) in the sample as a function of concentration of the inhibitor of HDAC6 deacetylase activity. One of skill in the art will understand that the amount and/or concentration of HDAC6 protein or activity present (such as the amount of acetyl HSP90, acetyl alpha-tubulin, AR, PSA, or TMPRSS2-ERG present) in the sample obtained from the subject can be measured at any concentration of the inhibitor of HDAC6 deacetylase activity or any number of concentrations of the inhibitor of HDAC6 deacetylase activity and that the concentrations given above are exemplary.

In some examples, the sample, such as a tumor sample obtained from a subject, is contacted with an inhibitor of HDAC6 deacetylase activity for at least about 1 second, such as at least about 5 seconds, at least about 10 seconds, at least about 30 seconds, at least about 1 minute, at least about 5 minutes, at least about 10 minutes, at least about 30 minutes, at least about 60 minutes, at least about 120 minutes, at least about 3 hours, at least about 6 hours, at least about 12 hours, or even about 24 hours or more. One of skill in the art will understand that a sample, such as a sample obtained from a subject can be contacted with an inhibitor of HDAC6 deacetylase activity for any amount of time and that periods given above are exemplary. In some examples, multiple time points are determined. For example, following contact with the inhibitor of HDAC6 deacetylase activity, the amount HDAC6 protein or activity present in the sample (such as the amount of HSP90 or alpha-tubulin acetylation or the amount of AR, PSA, or TMPRSS2-ERG present in the sample), such as a tumor sample obtained from a subject, is measured at various time points (for example to determine a time course a reduction in amount of HDAC6 protein or activity (such as to determine increase in the amount of HSP90 or alpha-tubulin acetylation or to determine reduction in the amount of AR, PSA, or TMPRSS2-ERG) in response to contact with the inhibitor of HDAC6 deacetylase activity), such as one more of about 10 seconds, at about 30 seconds, at about 1 minute, at about 5 minutes, at about 10 minutes, at about 30 minutes, at about 60 minutes, at about 2 hours, at least 24 hours, or even at least about 72 hours after contact with the inhibitor of HDAC6 deacetylase activity. One of skill in the art will understand that the amount of HDAC6 (or the amount of HSP90 acetylation, alpha-tubulin acetylation or the amount of AR, PSA, or TMPRSS2-ERG) can be measured at any time point or number of time points and that the time points given above are exemplary.

The methods disclosed herein are equally applicable to any cancer in which HDAC6 protein is found to play a role. Thus, the cancer can be any cancer of interest. For example the cancer can be a hematological cancer, such as a leukemia, including an acute leukemia (such as acute lymphocytic leukemia, acute myelocytic leukemia, acute myelogenous leukemia and myeloblastic, promyelocytic, myelomonocytic, monocytic and erythroleukemia), a chronic leukemia (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, or myelodysplasia. The cancer can also be a solid cancer, such as a sarcoma or a carcinoma, including a fibrosarcoma, myxosarcoma, liposarcoma, chondrosarcoma, osteogenic sarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). In some examples the cancer is breast cancer, CML, or GIST. In particular examples, the cancer is prostate cancer.

Acetylation of HSP90 and alpha-tubulin measured routine methods. For example, antibodies that are specific for the acetylated forms of such proteins (e.g., acetyl HSP90 and acetyl alpha-tubulin) can be used to detect the acetylated forms using standard immunological methods (such as those described below). Examples of antibodies that specifically bind acetylated alpha-tubulin protein include those available from ABCAM® catalogue no. ab24610 and Sigma, catalog number T6793. If antibodies are not available for the specific acetylated form of the protein, the proteins can be immunoprecipated (for example using an anti-acetyl lysine antibody, for example catalog no. ab76 from ABCAM, catalog no. 9441L from Cell Signaling Technology, Inc., Danvers, Mass., or catalog no. 05-515 from Upstate, Billerica, Mass.) and detected using Western blotting with an antibody specific for the protein (such as HSP90) to distinguish acetylated from non-acetylated forms of the protein. In addition, acetylated proteins can be detected and distinguished from non-acetylated forms using mass spectrophotometry methods as discussed below.

HDAC6 protein and other proteins (such as AR, PSA, or TMPRSS2-ERG protein) can be detected through novel epitopes recognized by polyclonal and/or monoclonal antibodies used in ELISA assays, immunoblot assays, flow cytometric assays, immunohistochemical assays, radioimmuno assays, Western blot assays, an immunofluorescent assays, chemiluminescent assays and other polypeptide detection strategies (Wong et al., Cancer Res., 46: 6029-6033, 1986; Luwor et al., Cancer Res., 61: 5355-5361, 2001; Mishima et al., Cancer Res., 61: 5349-5354, 2001; Ijaz et al., J. Med. Virol., 63: 210-216, 2001). Generally these methods utilize antibodies, such as monoclonal or polyclonal antibodies.

HDAC6 protein and other proteins (such as HSP-90, alpha-tubulin, AR, PSA, or TMPRSS2-ERG protein) can contain one or more conservative amino acid substitutions, for example polymorphisms present in the population. “Conservative” amino acid substitutions are those substitutions that do not substantially affect or decrease an activity or antigenicity of HDAC6 or other protein. For example, a HDAC6 polypeptide disclosed herein can include at most about 1, at most about 2, at most about 5, and at most about 10, or at most about 15 conservative substitutions and specifically bind an antibody that binds the original HDAC6 polypeptide such as set forth in GENBANK® Accession Nos. NP_{—}006035 (Homo sapiens), XP_{—}855362.1 (Canis familiaris), XP_{—}591306.3 (Bos Taurus), XP_{—}228753.4 (Rattus norvegicus); and NP_{—}034543.21 (Mus musculus) as available Oct. 8, 2008, which are incorporated herein by reference in their entirety. Conservative variations also includes the use of a substituted amino acid in place of an unsubstituted parent amino acid, provided that antibodies raised antibodies raised to the substituted polypeptide also immunoreact with the unsubstituted polypeptide. Non-conservative substitutions are those that reduce an activity or antigenicity.

In some embodiments, HDAC6 protein is detected using, for example, a HDAC6 specific binding agent, which can be detectably labeled. Other proteins (such as acetyl HSP-90, acetyl alpha-tubulin, AR, PSA, or TMPRSS2-ERG protein) can be similarly detected with appropriate specific binding agents. In some embodiments, the specific binding agent is an antibody, such as a polyclonal or monoclonal antibody, the specifically binds HDAC6 protein or other protein (such as HSP-90, alpha-tubulin, AR, PSA, or TMPRSS2-ERG protein). Thus in certain embodiments, detecting a HDAC6 includes contacting a sample from the subject with a HDAC6 specific binding agent (such as an antibody that specifically binds HDAC6), detecting whether the binding agent is bound by the sample, and thereby measuring the amount of HDAC6 protein present in the sample. In certain embodiments, the HDAC6 specific binding agent is an antibody or an antibody fragment that specifically binds HDAC6. In one embodiment, the specific binding agent is a monoclonal or polyclonal antibody that specifically binds the HDAC6 polypeptide.

An antibody that specifically binds a HDAC6 protein or other protein (such as acetyl HSP-90, acetyl alpha-tubulin, AR, PSA, or TMPRSS2-ERG protein) typically binds with an affinity constant of at least 10⁷ M⁻¹, such as at least 10⁸ M⁻¹ at least 5×10⁸ M⁻¹ or at least 10⁹ M⁻¹. All of these antibodies are of use in the methods disclosed herein.

The preparation of polyclonal antibodies is well known to those skilled in the art. See, for example, Green et al., “Production of Polyclonal Antisera,” in: Immunochemical Protocols pages 1-5, Manson, ed., Humana Press 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition including an antigen or a cell of interest, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

Antibodies include intact molecules as well as functional fragments thereof, such as Fab, F(ab′)₂, and Fv which are capable of binding the epitopic determinant. These antibody fragments retain some ability to selectively bind with their antigen. Methods of making these fragments are known in the art. (See for example, Harlow and Lane, Antibodies: A Laboratory Manual, Cold Spring Harbor Laboratory, New York, 1988).

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

Examples of antibodies that specifically bind HDAC6 protein include those available from ABCAM® catalogue nos. ab61058, ab1440, ab11970, ab12173, ab53099, ab56926, ab61173 and ab47181; and Santa Cruz Biotechnology, Inc. catalogue nos. sc-28386, sc-11420, and sc-5258. Examples of antibodies that specifically bind AR protein include those available from ABCAM® catalogue nos. ab2742, ab9474, and ab3509; and Santa Cruz Biotechnology, Inc. catalogue nos. sc-816, sc-52309, and sc-70375. Examples of antibodies that specifically bind PSA protein include those available from ABCAM® catalogue nos. ab403, ab63590 and ab9537; and Santa Cruz Biotechnology, Inc. catalogue nos. sc-80304, sc-7638, and sc-69664. Examples of antibodies that specifically bind TMPRSS2-ERG protein include those available from ABCAM® catalogue nos. ab56111, ab82146 and ab56110; and Santa Cruz Biotechnology, Inc. catalogue nos. sc-33533, sc-19686, and sc-101847. One skilled in the art will appreciate that there are other commercial sources for antibodies to these proteins.

The antibodies used in the methods disclosed herein can be labeled. The enzymes that can be conjugated to the antibodies include, but are not limited to, alkaline phosphatase, peroxidase, urease and B-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes: Handbook of Fluorescent Probes and Research Chemicals (1992-1994). The metal compounds that can be conjugated to the antibodies include, but are not limited to, ferritin, colloidal gold, and particularly colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids including any radionucleotides, including but not limited to, ¹⁴C, ³H and ³⁵S.

HDAC6 polypeptides or other proteins (such as acetylated HSP-90, acetylated alpha-tubulin, AR, PSA, or TMPRSS2-ERG protein) also can be detected by mass spectrometry assays for example coupled to immunaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of tumor derived proteins separated by two-dimensional polyacrylamide gel electrophoresis (2D-PAGE) (Kiernan et al., Anal. Biochem., 301: 49-56, 2002; Poutanen et al., Mass Spectrom., 15: 1685-1692, 2001).

The presence of a HDAC6 polypeptide or other proteins (such as acetylated HSP-90, acetylated alpha-tubulin, AR, PSA, or TMPRSS2-ERG protein) can be determined with multiple specific binding agents, such as one, two, three, or more specific binding agents. Thus, the methods can utilize more than one antibody. In some embodiments, one of the antibodies is attached to a solid support, such as a multiwell plate (such as, a microtiter plate), bead, membrane or the like. In practice, microtiter plates may conveniently be utilized as the solid phase. The surfaces may be prepared in advance, stored, and shipped to another location(s). However, antibody reactions also can be conducted in a liquid phase.

In some examples, the control is the amount of HDAC6 or other protein (such as acetylated HSP-90, acetylated alpha-tubulin, AR, PSA, or TMPRSS2-ERG protein) in a sample not contacted with an inhibitor of HDAC6 deacetylase activity, a sample of non-cancerous cells, a sample obtained from the subject from an earlier time point, or a combination thereof. In some examples, a control is a value indicative of the basal amount of HDAC6, and one or more of acetylated HSP-90, acetylated alpha-tubulin, AR, PSA, or TMPRSS2-ERG protein. In some examples, the control is a statistical value, for example measured from multiple control samples. In some embodiments, the difference between the amount of HDAC6 protein (or acetylated HSP-90, acetylated alpha-tubulin, AR, PSA, or TMPRSS2-ERG protein) in the cell contacted with the an inhibitor of HDAC6 deacetylase activity relative to a control is at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500% or even greater then 500%.

### B. Methods of Monitoring Disease in a Subject

The methods disclosed herein are useful for monitoring the effectiveness of a treatment for cancer in a subject, for example prostate cancer treatment. In some embodiments, the methods can include detecting an amount of HDAC6 protein or activity present in a sample obtained from a subject (such as a tumor sample) that is undergoing a treatment for cancer and comparing that amount with a control (such as an amount of HDAC6 protein or activity present in a normal prostate sample, for example a reference value or range of values representing the expected HDAC6 protein or activity levels in a normal prostate cell or sample). In a specific non-limiting embodiment, the cancer is prostate cancer. In some examples the treatment is treatment with an HDAC6 inhibitor, for example sulforaphane. The control can be but is not limited to the any of the controls listed in the preceding section.

A decrease in the amount of HDAC6 protein relative to a control (such as an amount of HDAC6 protein present in a normal prostate sample, for example a reference value or range of values representing the expected HDAC6 protein levels in a normal prostate cell or sample) indicates that the subject is responding to the cancer treatment. For example, a reduction of at least 10%, at least 20%, at least 30%, at least 50%, at least 75%, at least 80%, at least 90%, at least 95%, at least 98% or even at least 99%, indicates that the subject (such as a human subject with prostate cancer) is responding to for treatment with an inhibitor of HDAC6 deacetylase activity, such as sulforaphane, tubacin, belinostat, or other inhibitor known in the art.

In contrast, an increase or maintenance in the amount of HDAC6 protein relative to the control (such as an amount of HDAC6 protein present in a normal prostate sample, for example a reference value or range of values representing the expected HDAC6 protein levels in a normal prostate cell or sample) indicates that the subject is not responding to the cancer treatment, for example a treatment with an inhibitor HDAC6 deacetylase activity, such as sulforaphane, tubacin, belinostat or other inhibitor known in the art. For example, an increase of at least 10%, at least 20%, at least 30%, at least 50%, at least 75%, at least 80%, at least 90%, at least 100%, at least 200% or even at least 500%, indicates that the subject (such as a human subject with prostate cancer) is not responding to treatment with an inhibitor of HDAC6 deacetylase activity.

In some examples, HDAC6 activity is measured in the subject's sample, for example in combination with detecting HDAC6 protein levels. Exemplary HDAC6 activities that can be measured include detection of levels of acetyl HSP90, acetyl alpha-tubulin, AR, PSA, or TMPRSS2-ERG protein (for example using antibodies specific for these proteins). An increase in HSP90 or alpha-tubulin acetylation, or a decrease in levels of AR, PSA, or TMPRSS2-ERG proteins, relative to a control, indicates that the subject is responding to treatment with an inhibitor of HDAC6 deacetylase activity, such as sulforaphane, tubacin, belinostat or other inhibitor known in the art. For example, an increase of acetyl HSP90 or acetyl alpha-tubulin of at least 10%, at least 20%, at least 30%, at least 50%, at least 75%, at least 80%, at least 90%, at least 95%, at least 98%, at least 99%, at least 200%, at least 300%, or even at least 500% relative to the control, or a reduction of in AR protein, PSA protein, or TMPRSS2-ERG protein of at least 10%, at least 20%, at least 30%, at least 50%, at least 75%, at least 80%, at least 90%, at least 95%, at least 98% or even at least 99%, relative to the control, indicates that the subject (such as a human subject with prostate cancer) is responding to the cancer treatment (such as treatment with an inhibitor of HDAC6 deacetylase activity). In contrast, an increase in AR protein, PSA protein, or TMPRSS2-ERG protein of at least 10%, at least 20%, at least 30%, at least 50%, at least 75%, at least 80%, at least 90%, at least 95%, at least 98%, at least 99%, at least 200%, at least 300%, or even at least 500%, or a reduction of acetyl HSP90 or acetyl alpha-tubulin of at least 10%, at least 20%, at least 30%, at least 50%, at least 75%, at least 80%, at least 90%, at least 95%, at least 98% or even at least 99%, relative to the control, indicates that the subject (such as a human subject with prostate cancer) is not responding to the cancer treatment (such as treatment with an inhibitor of HDAC6 deacetylase activity).

In some embodiments, methods of monitoring disease progression in a subject involve detecting the amount of HDAC6 protein or activity, for example detecting HDAC6 protein in combination with detecting one or more of acetyl HSP90, acetyl alpha-tubulin, AR protein, PSA protein, or TMPRSS2-ERG protein in a sample, such as a tumor sample, obtained from a subject at a first time point and comparing that amount with the amount of HDAC6 protein (and as appropriate acetyl HSP90, acetyl alpha-tubulin, AR protein, PSA protein, or TMPRSS2-ERG protein) detected in a sample, such as a tumor sample, obtained from a subject at a second time point, for example a later time point. If an increase in the amount of HDAC6 protein (and as appropriate AR protein, PSA protein, or TMPRSS2-ERG protein) is observed at the second time point (for example an increase of at least 10%, at least 20%, at least 30%, at least 50%, at least 75%, at least 80%, at least 90%, at least 100%, at least 200% or even at least 500%), or if a decrease in acetyl HSP90 or acetyl alpha-tubulin is observed at the second time point (for example a reduction of at least 10%, at least 20%, at least 30%, at least 50%, at least 75%, at least 80%, at least 90%, at least 95%, at least 98% or even at least 99%), the subject is showing signs of disease progression, and the subject may not benefit from continued treatment (for example with an inhibitor HDAC6 deacetylase activity, such as sulforaphane, tubacin, or belinostat). Conversely, if a decrease in the amount of HDAC6 (and as appropriate AR, PSA, or TMPRSS2-ERG protein) at the second time point is observed (for example a reduction of at least 10%, at least 20%, at least 30%, at least 50%, at least 75%, at least 80%, at least 90%, at least 95%, at least 98% or even at least 99%), or if an increase in HSP90 or alpha-tubulin acetylation is observed at the second time point (for example an increase of at least 10%, at least 20%, at least 30%, at least 50%, at least 75%, at least 80%, at least 90%, at least 100%, at least 200% or even at least 500%) the subject is showing signs of disease remission, and the subject would benefit from continued treatment (for example with an inhibitor HDAC6 deacetylase activity, such as sulforaphane, tubacin, or belinostat). In some embodiments the subject is monitored for progression of prostate cancer.

Also encompassed by this disclosure are methods for selecting a treatment regimen or therapy for the prevention, reduction, or inhibition of cancer, such as prostate cancer. In some examples, these methods involve detecting a decrease in the amount of HDAC6 protein in a sample, such as tumor sample, obtained from a subject relative to a control (such as an amount of HDAC6 protein present in a normal prostate sample, for example a reference value or range of values representing the expected HDAC6 protein levels in a normal prostate cell or sample) in response to a treatment. If such decrease is detected (for example a reduction of at least 10%, at least 20%, at least 30%, at least 50%, at least 75%, at least 80%, at least 90%, at least 95%, at least 98% or even at least 99%), the treatment is selected to prevent or reduce cancer or to delay the onset of cancer, such as prostate cancer. The subject then can be treated in accordance with this selection.

In some examples, HDAC6 activity is measured, for example in combination with measuring HDAC6 protein levels. For example, one or more of the following can be also detected as indicators of HDAC6 activity: acetyl HSP90, acetyl alpha-tubulin, AR protein, PSA protein, or TMPRSS2-ERG protein (for example using antibodies specific for these proteins). An increase in detected HSP90 or alpha-tubulin acetylation (for example increases in acetyl HSP90 or acetyl alpha-tubulin), or a decrease in protein levels of AR, PSA, or TMPRSS2-ERG, relative to a control, indicates that the treatment can be selected to prevent or reduce cancer or to delay the onset of cancer, such as prostate cancer. The subject then can be treated in accordance with this selection. For example, an increase of detected acetyl HSP90 or acetyl alpha-tubulin of at least 10%, at least 20%, at least 30%, at least 50%, at least 75%, at least 80%, at least 90%, at least 95%, at least 98%, at least 99%, at least 200%, at least 300%, or even at least 500% relative to the control, or a reduction of in AR, PSA, or TMPRSS2-ERG of at least 10%, at least 20%, at least 30%, at least 50%, at least 75%, at least 80%, at least 90%, at least 95%, at least 98% or even at least 99%, relative to the control, indicates that that the treatment can be selected to prevent or reduce cancer or to delay the onset of cancer, such as prostate cancer.

Such treatments include without limitation the use of chemotherapeutic agents, immunotherapeutic agents, radiotherapy, surgical intervention, inhibitors of HDAC6 deacetylase activity (such as sulforaphane, tubacin, or belinostat) or combinations thereof.

### C. Identification of Therapeutic Compounds

Detecting a decrease in the amount of HDAC6 protein or activity can be used to identify compounds that are useful in treating, reducing, or preventing cancer, such as prostate cancer. Thus, this disclosure also relates methods for identifying agents for the treatment of cancer, such as prostate cancer. The methods for identifying compounds useful for treating such cancer involve contacting at least one cell with one or more test agents, detecting an amount of HDAC6 protein in the cell, and comparing the amount of HDAC6 protein detected to a control (such as an amount of HDAC6 protein present in a normal prostate sample, for example a reference value or range of values representing the expected HDAC6 protein levels in a normal prostate cell or sample) to determine if the test agent reduces the amount of HDAC6 protein. A test agent that reduces the amount of HDAC6 protein relative to the control (for example a reduction of at least 10%, at least 20%, at least 30%, at least 50%, at least 75%, at least 80%, at least 90%, at least 95%, at least 98% or even at least 99%) is identified as an agent inhibits cancer.

In some examples, HDAC6 activity is measured, for example in combination with measuring HDAC6 protein levels. For example, one or more of the following can be also detected as indicators of HDAC6 activity: acetyl HSP90, acetyl alpha-tubulin, AR protein, PSA protein, or TMPRSS2-ERG protein. An increase in HSP90 or alpha-tubulin acetylation (for example detection of increased amounts of ac) or a decrease in levels of AR, PSA, or TMPRSS2-ERG proteins, relative to a control, identifies the test agent as an agent inhibits cancer. For example, an increase of HSP90 or alpha-tubulin acetylation of at least 10%, at least 20%, at least 30%, at least 50%, at least 75%, at least 80%, at least 90%, at least 95%, at least 98%, at least 99%, at least 200%, at least 300%, or even at least 500% relative to the control, or a reduction of in AR protein, PSA protein, or TMPRSS2-ERG protein of at least 10%, at least 20%, at least 30%, at least 50%, at least 75%, at least 80%, at least 90%, at least 95%, at least 98% or even at least 99%, relative to the control, identifies the test agent as an agent inhibits cancer. In contrast, an increase in AR protein, PSA protein, or TMPRSS2-ERG protein of at least 10%, at least 20%, at least 30%, at least 50%, at least 75%, at least 80%, at least 90%, at least 95%, at least 98%, at least 99%, at least 200%, at least 300%, or even at least 500%, or a reduction of HSP90 or alpha-tubulin acetylation of at least 10%, at least 20%, at least 30%, at least 50%, at least 75%, at least 80%, at least 90%, at least 95%, at least 98% or even at least 99%, relative to the control, indicates that the subject (such as a human subject with prostate cancer) is not responding to the cancer treatment (such as treatment with an inhibitor of HDAC6 deacetylase activity), and in some examples an alternative treatment is selected.

In some examples, the cell is contacted with a test agent at a concentration of about 1 picomolar to about 100 mmolar, such as a concentration of test agent of about 1 picomolar, about 10 picomolar, about 100 picomolar, about 1 nanomolar, about 10 nanomolar, about 100 nanomolar, about 1 micromolar, about 10 micromolar, about 100 micromolar, 1 millimolar, about 10 millimolar, or even about 100 millimolar. In some examples, the cell is contacted with different concentrations of the test agent, for example to determine the amount of HDAC6 protein (or other protein such as acetyl HSP90, acetyl alpha-tubulin, AR protein, PSA protein, or TMPRSS2-ERG protein) as a function of test agent concentration. One of skill in the art will understand that the amount and/or concentration of HDAC6 protein (or other protein such as acetyl HSP90, acetyl alpha-tubulin, AR protein, PSA protein, or TMPRSS2-ERG protein) can be measured at any concentration of test agent or any number of concentrations of test agent and that the concentrations given above are exemplary.

In some examples, the cell is contacted with the test agent for at least about 1 second, such as at least about 5 seconds, at least about 10 seconds, at least about 30 seconds, at least about 1 minute, at least about 5 minutes, at least about 10 minutes, at least about 30 minutes, at least about 60 minutes, at least about 120 minutes, at least about 3 hours, at least about 6 hours, at least about 12 hours, or even about 24 hours or more. One of skill in the art will understand that a cell can be contacted with the test agent for any amount of time and that periods given above are exemplary.

In some examples, multiple time points are determined. For example, following incubation with the one or more test agents, the amount of HDAC6 protein (or other protein such as acetyl HSP90, acetyl alpha-tubulin, AR protein, PSA protein, or TMPRSS2-ERG protein) is measured at various time points (for example to determine a time course for the decrease in the amount of HDAC6 or other protein in response to contact with a test agent), such at about 10 seconds, at about 30 seconds, at about 1 minute, at about 5 minutes, at about 10 minutes, at about 30 minutes, at about 60 minutes, at about 2 hours, or even at least 24 hours after contact with the test agent. One of skill in the art will understand that the amount and/or concentration of HDAC6 protein (or other protein such as acetyl HSP90, acetyl alpha-tubulin, AR protein, PSA protein, or TMPRSS2-ERG protein) can be measured at any time point or number of time points and that the time points given above are exemplary.

In some embodiments, a test agent can induce a statistically significant difference in the amount of HDAC6 protein (or other protein such as acetyl HSP90, acetyl alpha-tubulin, AR protein, PSA protein, or TMPRSS2-ERG protein) in a cell contacted with the test agent, as compared to the control, such as a cell not contacted with the test agent (such as a cell contacted with carrier alone). In some embodiments, the difference between the amount of HDAC6 protein (or other protein such as acetyl HSP90, acetyl alpha-tubulin, AR protein, PSA protein, or TMPRSS2-ERG protein) in the cell contacted with the test agent relative to a control is at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500% or even greater then 500%.

In some examples, the cell contacted with the test agent is a primary cell obtained from a mammalian or primate subject, for example a prostate cell. In other example, an established cell line is used, such as an LNCaP cell or a VCaP cell, or even a combination of such cells. Exemplary test agents are given below. In some example, a high throughput technique is used, so that multiple test agents can be screened, for example in parallel. Any method of detecting HDAC6 protein (or other protein such as acetyl HSP90, acetyl alpha-tubulin, AR protein, PSA protein, or TMPRSS2-ERG protein) can be used. Some particularly convenient examples are immunohistochemical assays, a radioimmuno assays, Western blot assays, immunofluorescent assays, enzyme immunoassasys, chemiluminescent assays, or a mass spectrometry assays.

A variety of controls can be used with the disclosed methods. In some examples, the control is a standard value, for example a value that indicates that basal level of HDAC6 protein in a cell. In other examples, a control is the amount of HDAC6 protein in a cell that has not been contacted with a test agent.

By way of example, a test compound is applied to a cell, for instance a test cell, which is monitored for the amount of HDAC6 protein or activity in the cell. HDAC6 protein (or other protein such as acetyl HSP90, acetyl alpha-tubulin, AR protein, PSA protein, or TMPRSS2-ERG protein) in the contacted test cell is compared to the equivalent measurement from a test cell in the absence of the test compound. Compounds that reduce the amount of HDAC6 protein (and in some examples reduce the amount of AR protein, PSA protein, or TMPRSS2-ERG protein and/or increase the amount of acetyl HSP90 or acetyl alpha-tubulin), are selected for further characterization to determine toxicity, bioavailability, stability, and the like. Additionally, the activity of the selected compound to inhibit growth of cancer, such as prostate cancer is typically evaluated in vitro and/or in vivo to confirm biological activity. Such identified compounds are useful in treating, reducing, or preventing cancer or development or progression of cancer.

### D. Exemplary Test Agents

An “agent” is any substance or any combination of substances that is useful for achieving an end or result. The agents identified using the methods disclosed herein can be of use for treating and/or preventing cancer, such as prostate cancer. Any agent that has potential (whether or not ultimately realized) to reduce the amount of HDAC6 protein (and in some examples also reduce the amount of AR protein, PSA protein, or TMPRSS2-ERG protein and/or increase the amount of acetyl HSP90 or acetyl alpha-tubulin) can be tested using the methods of this disclosure.

Exemplary agents include, but are not limited to, peptides such as, soluble peptides, including but not limited to members of random peptide libraries (see, for example Lam et al., Nature, 354:82-84, 1991; Houghten et al., Nature, 354:84-86, 1991), and combinatorial chemistry-derived molecular library made of D-and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids. In some examples, a test agent is a known anti-neoplastic agent.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (for example the number of amino acids in a polypeptide compound). Millions of chemical compounds can be synthesized through such combinatorial mixing of chemical building blocks.

Appropriate test agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Preparation and screening of combinatorial libraries is well known to those of skill in the art. Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), (see, e.g., Lam et al., Nature, 354:82-84, 1991; Houghten et al., Nature, 354:84-86, 1991), and combinatorial chemistry-derived molecular library made of D-and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), small organic or inorganic molecules (such as, so-called natural products or members of chemical combinatorial libraries), molecular complexes (such as protein complexes), or nucleic acids, encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, Jan 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997).

Devices for the preparation of combinatorial libraries are also commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, for example, ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Libraries can include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one example, the methods can involve providing a combinatorial chemical or peptide library containing a large number of potential therapeutic compounds. Such combinatorial libraries are then screened by the methods disclosed herein to identify those library members (particularly chemical species or subclasses) that display a desired characteristic activity.

The compounds identified using the methods disclosed herein can serve as conventional “lead compounds” or can themselves be used as potential or actual therapeutics. In some instances, pools of candidate test agents can be identified and further screened to determine which individual or subpools of test agents in the collective have a desired activity.

Control reactions such as those described in the sections above can be performed in combination with the libraries. Such optional control reactions are appropriate and can increase the reliability of the screening.

### E. Kits and High Throughput Systems

This disclosure also provides kits for measuring the levels of HDAC6 protein in a sample, such as sample obtained from a subject, or cells for in identifying a test agent for the treatment of cancer, such as prostate cancer. The kits include antibodies that specifically bind HDAC6 protein. In some examples, the kits further include antibodies that recognize other protein, such as one or more of AR, PSA, and TMPRSS2-ERG. In some examples, the kits further include antibodies that recognize one or more of acetylated or non-acetylated HSP90 or alpha-tubulin. In one example, the kit includes a graph or table showing expected values or ranges of values of HDAC6 protein (and in some examples one or more of acetyl HSP90, acetyl alpha-tubulin, AR, PSA, and TMPRSS2-ERG protein) expected in a normal prostate cell. In some examples, kits further include control samples, such as particular quantities of HDAC6 protein (and in some examples one or more of AR, PSA, TMPRSS2-ERG, acylated HSP90 or acylated alpha-tubulin protein).

The kits may further include additional components such as instructional materials and additional reagents, for example specific binding agents, such as secondary antibodies (for example antibodies that specifically bind the primary antibodies that specifically bind HDAC6 protein or other protein) or a means for labeling antibodies. The kits may also include additional components to facilitate the particular application for which the kit is designed (for example microtiter plates). Such kits and appropriate contents are well known to those of skill in the art. The instructional materials may be written, in an electronic form (such as a computer diskette or compact disk) or may be visual (such as video files).

This disclosure also provides integrated systems for high-throughput screening, for example of samples obtained from subjects, or cellular systems for identifying agent for use in treating cancer. The systems typically include a robotic armature that transfers fluid from a source to a destination, a controller that controls the robotic armature, a tag detector, a data storage unit that records tag detection, and an assay component such as a microtiter dish comprising a well having a reaction mixture for example media.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous assays, for example to assay for the effect of one or more test agents or an inhibitor of HDAC6 deacetylase activity on the amount of HDAC6 protein in a sample, such as cells or samples obtained from a subject, such as a tumor sample (for example a prostate tumor sample).

A variety of commercially available peripheral equipment and software is available for digitizing, storing and analyzing a digitized video or digitized optical image, e.g., using PC (Intelx86 or Pentium chip-compatible DOS™ 0S2™ WINDOWS™, WINDOWS NT™ or WINDOWS95™ based computers), MACINTOSH™, or UNIX based (for example, a SUN™, a SGI™, or other work station) computers.

The following examples are provided to illustrate certain particular features and/or embodiments. These examples should not be construed to limit the invention to the particular features or embodiments described.

### EXAMPLES

### Example 1

### Sulforaphane Treatment Increases HSP90 Acetylation and Leads to Dissociation of AR from HSP90

This example describes methods used to determine that HSP90 acetylation is increased by sulforaphane treatment. Certain HDAC inhibitors inhibit protein deacetylases such as HDAC6 whose targets include the HSP90 protein. Deacetylated HSP90 stabilizes a key prostate cancer protein androgen receptor (AR). Thus, the effect of sulforaphane (an inhibitor of HDAC6) on HSP90 acetylation was examined.

LNCaP and VCaP cells were grown according to ATCC instructions. Sulforaphane (SFN, #S4441 Sigma, St. Louis, Mo.) was re-suspended in DMSO. Vehicle-treated cells received the respective vehicle controls Immunoprecipitations were carried out after treatment with sulforaphane (20 μM) or vehicle for 4 hours followed by a Western blot for HSP90 and AR. Enrichment was quantified for each immunoprecipitation. Inputs were probed with the indicated antibodies by Western blot and were quantified.

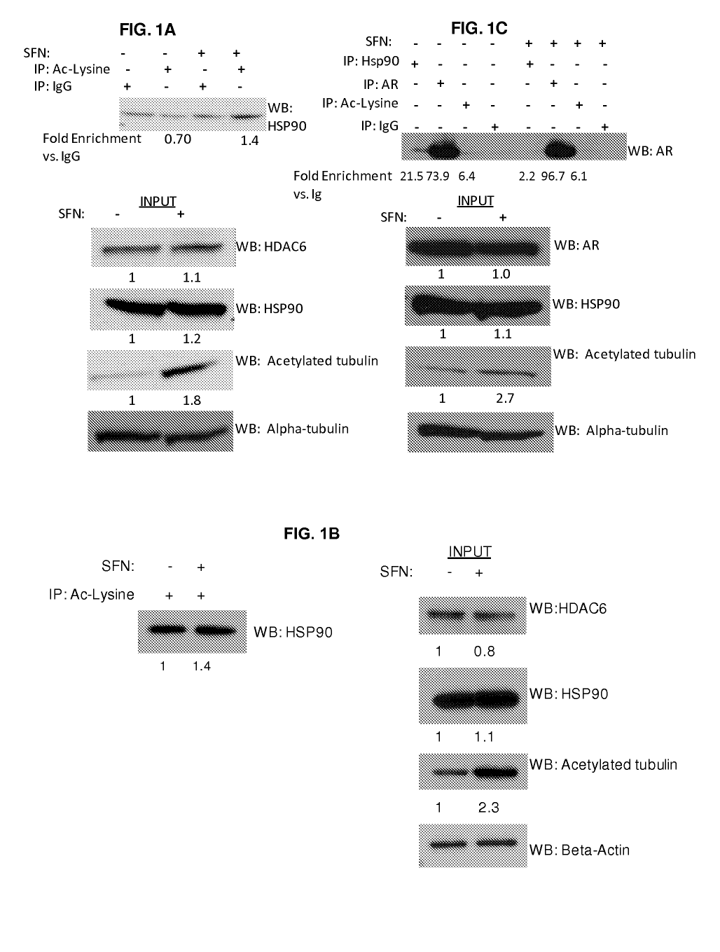

Immunoprecipitation and immunoblotting were performed as described (Qian et al., Clin. Cancer Res. 12:634-42, 2006). The following antibodies used in immunoblotting or immunoprecipitations (indicated dilutions are for immunoblotting): AR (1:1000, #06-680 Upstate, Billerica, Mass.); acetylated lysine (#05-515 Upstate, Billerica, Mass.); acetylated tubulin (1:2000, #T6793 Sigma, St. Louis, Mo.); alpha-tubulin (1:10,000, #T6074 Sigma, St. Louis, Mo.); beta-actin (1:5000, #A5441 Sigma, St. Louis, Mo.); ERG-1/2/3, (1:5000, #sc-354X Santa Cruz, Santa Cruz, Calif.); FLAG epitope (#F7425 Sigma, St. Louis, Mo.); GAPDH (1:5000, #sc-32233 Santa Cruz, Santa Cruz, Calif.); HDAC6 (1:1000, #sc-11420 Santa Cruz); HSP90 (1:1000, #SPA-840 Stressgen, Ann Arbor, Mich.); mouse IgG (#sc-2025 Santa Cruz, Santa Cruz, Calif.). The following secondary antibodies were used: ECL-conjugated anti-mouse IgG, (1:5000, #A6782 Sigma, St. Louis, Mo.); ECL-conjugated anti-rabbit IgG (1:3000, #NA934 Amersham, Piscataway, N.J. or 1:10,000, #sc-2004 Santa Cruz, Santa Cruz, Calif.). X-ray exposures were scanned as uncompressed images and bands were quantified by densitometry with Quantity One software (Bio-Rad, Hercules, Calif.). Intensity values were normalized to those of the endogenous controls. The vehicle sample was set to 1. Enrichment was calculated as: (intensity IP/intensity input)/(intensity IgG/intensity input) or intensity IP/input (FIG. 1B).

LNCaP cells, an AR-expressing prostate cancer cell line, were treated with sulforaphane or vehicle control. These cells showed increased enrichment of acetylated lysines on the HSP90 protein versus vehicle-treated cells as early as four hours after treatment (FIG. 1A). In the input, whole cell lysate samples, levels of HSP90, HDAC6, the only known HSP90 protein deacetylase, and alpha-tubulin were similar between vehicle and sulforaphane-treated cells whereas the sulforaphane-treated samples have increased levels of acetylated tubulin, a HDAC6 target, further indicating inhibition of protein deacetylation at this time point (FIG. 1A). Similar results were seen in VCaP prostate cancer cells (FIG. 1B).

It was next determined whether, in addition to enhancing HSP90 acetylation, treatment with sulforaphane has functional consequences on HSP90 chaperone function on the AR protein. As shown in FIG. 1C, with a 4 hour sulforaphane treatment, as opposed to vehicle treatment, the interaction between HSP90 and AR was disrupted. The upper band of the gel image shown in FIG. 1C represents the phosphorylated AR protein while the bottom band represents unphosphorylated AR protein. The immunoprecipitation with AR followed by Western blotting for AR shows similar levels between vehicle (lane 2) and sulforaphane-treated (lane 6) cells indicating similar amounts of AR protein in these cells in the setting of decreased HSP90-AR interaction with sulforaphane treatment (lanes 1 and 5). Levels of acetylation of the AR were similar between the cells treated with vehicle or sulforaphane (lanes 3 and 7). In the input samples, whole cell lysate samples, levels of HSP90, AR (same migration pattern to top band seen in IP), and tubulin were similar between vehicle and sulforaphane-treated cells whereas the sulforaphane-treated samples have increased levels of acetylated alpha-tubulin, again indicating inhibition of protein deacetylation of an HDAC6 target at this same time point (FIG. 1C).

These results demonstrate that inhibition of HDAC6 activity (using the HDAC inhibitor sulforaphane) leads to an increase in the levels of acetylated HSP90 and a concomitant dissociation of AR from HSP90.

### Example 2

### Sulforaphane Treatment Lowers AR Protein Levels

This example demonstrates that inhibition of HDAC6 leads to AR protein ablation.

LNCaP cells were treated with sulforaphane in a dose-response (5 μM to 20 μM) and time-course (12 and 24 hours) manner to investigate the effect of HDAC6 inhibition on AR protein levels. Western blotting was performed as described in Example 1.

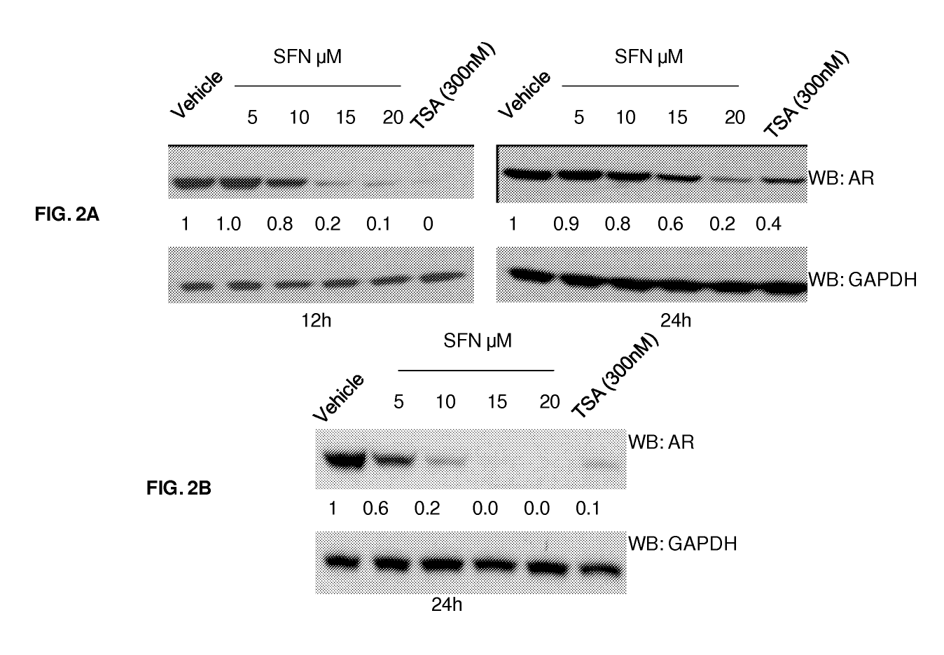

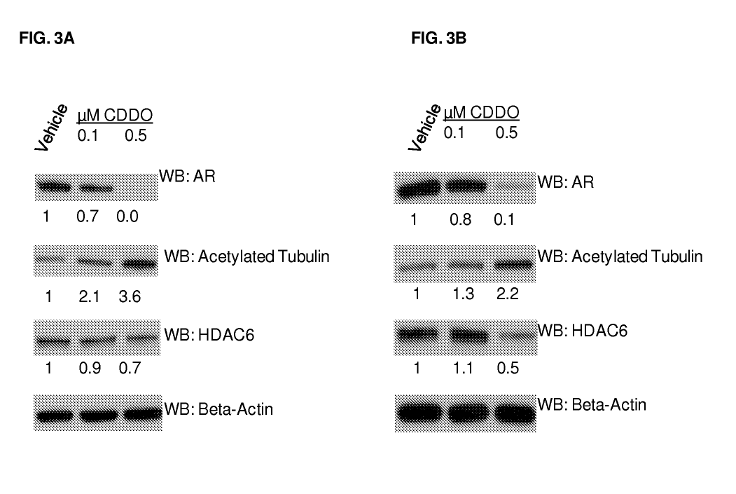

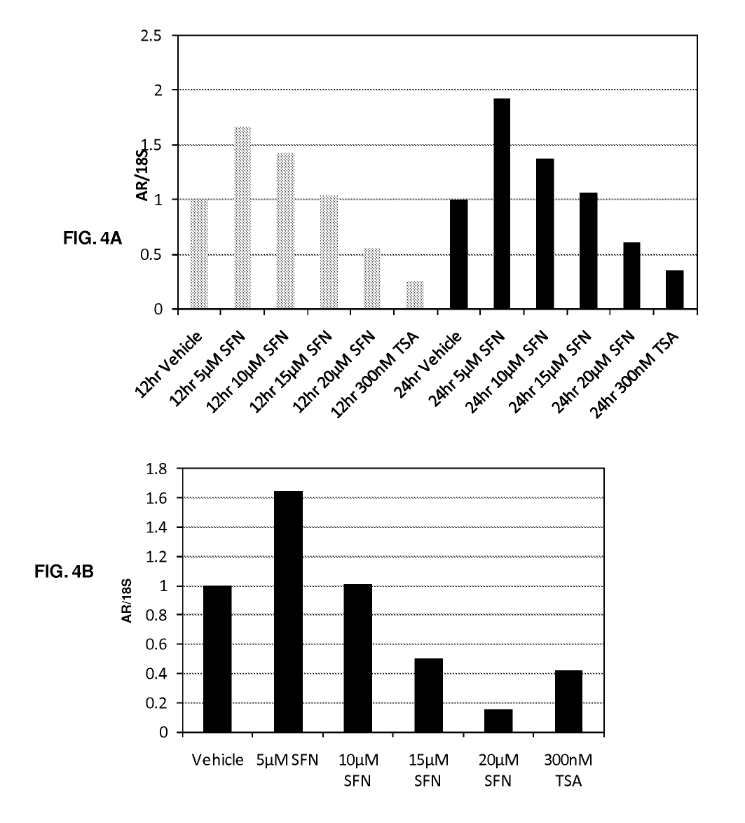

AR protein levels decreased by 12 hours after treatment for the highest doses of sulforaphane tested (10-20 μM) (FIG. 2A). In VCaP cells, at 24 hours, AR protein levels declined for all sulforaphane dose levels (FIG. 2B). Similar results were seen for both cell lines with the pan-HDAC inhibitor trichostatin A (TSA) and the triterpenoid CDDO-Imidazole (National Cancer Institute/DTP Open Chemical Repository) that, like sulforaphane, acts via the Nrf2-Keapl pathway (FIGS. 3A and 3B). For the 15 μM and 20 μM doses, this effect persisted even at 24 hours, in contrast to TSA, for which AR protein levels had already recovered. AR transcripts were not consistently suppressed by sulforaphane treatment, demonstrating that post-transcriptional mechanisms are involved (FIGS. 4A and B). Thus, across both AR-expressing cell lines, sulforaphane treatment reduced AR proteins levels.

While for most doses of sulforaphane, AR proteins levels decline without reductions in AR transcript levels, for the highest doses of sulforaphane (20 μM in LNCaP cells and 15-20 μM in VCaP cells) AR transcript levels also declined. It was also observed that the pan-HDAC inhibitor trichostatin A (TSA) also suppressed AR transcripts (FIGS. 2A and 2B). Consequently the effect of sulforaphane on AR levels is wholly post-translational, although there is clear rescue of AR levels with simultaneous sulforaphane and proteasome inhibitor treatment. High-dose sulforaphane treatment could lead to reduced AR transcription or enhanced AR transcript degradation.

### Example 3

### Sulforaphane Treatment Lowers AR Target Gene Expression Including ERG

This example demonstrates that HDAC6 inhibition with sulforaphane reduces expression of AR target genes.

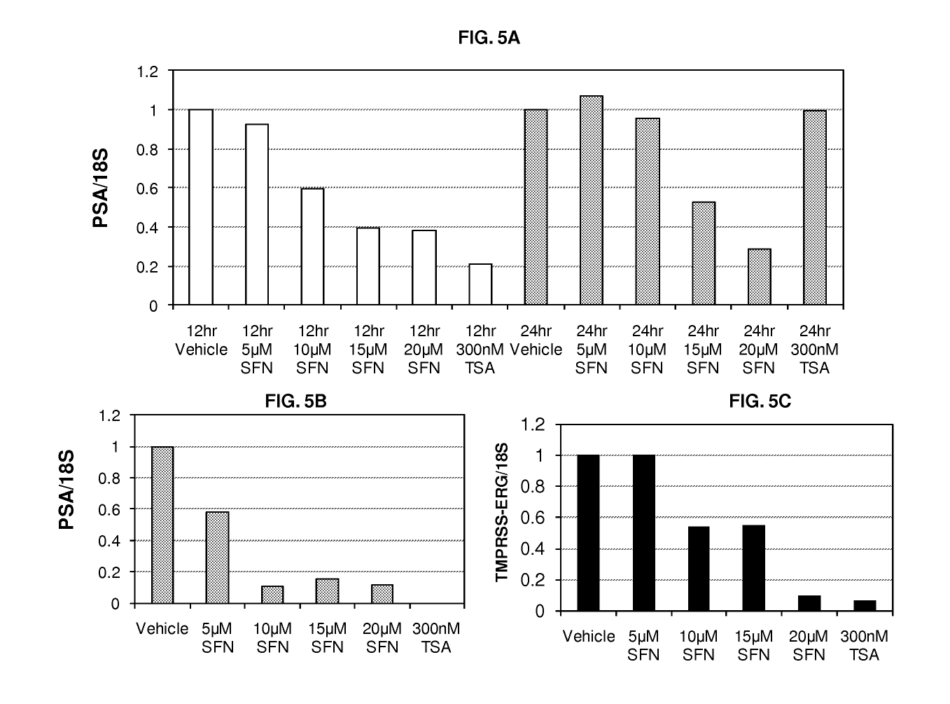

To determine the functional consequences of sulforaphane-mediated reduction in AR protein levels, real-time PCR was conducted for several AR target genes, including PSA and the TMPRSS2-ERG gene fusion present in VCaP cells (FIGS. 5A-C). Cells were lysed in Trizol (Invitrogen, Carlsbad, Calif.) and then purified with the RNAEasy Kit (Qiagen, Valencia, Calif.). One μg of RNA was reverse-transcribed using the Omniscript RT kit (Qiagen, Valencia, Calif.). Real-time PCR primer sequences and conditions were as follows. Taqman gene expression assays for AR (Hs00171172_m1), PSA (KLK3, Hs02576345_m1), HDAC6 (Hs00195869_m1), and 18S endogenous control (4319413E) were purchased from ABI (Foster City, Calif.). Primers and probe for TMPRSS2-ERG fusion were from IDT (Coralville, Iowa):

TMPRSS2-ERG QRTPCR Sense Primer:
(SEQ ID NO: 1)
CGCGAGCTAAGCAGGAG
TMPRSS2-ERG QRTPCR Anti-Sense Primer:
(SEQ ID NO: 2)
CGACTGGTCCTCACTCACAA
TMPRSS2-ERG QRTPCR Probe:
(SEQ ID NO: 3)
FAM5′-CGCGGCAGGAAGCCTTATCAG-3′TAMRA

The real-time PCR program consisted of: 50° C. for 2 minutes for 1 cycle; 95° C. for 10 minutes for 1 cycle; 95° C. for 15 seconds and 60° C. for 15 seconds for a total of 40 cycles. Standard curves were generated by measuring expression of target genes and 18S rRNA in serial dilutions of a mock-treated control. All samples were run in triplicate. The vehicle-treated sample was set to 1.

There was a dose-dependent reduction in gene expression in both cell lines (FIGS. 5A-C). Similar results were seen with TSA. Thus, there is a high concordance between reduced AR protein levels and reduced AR target gene expression in prostate cancer cells.

### Example 4

### Sulforaphane Treatment Reduces AR Occupancy at its AREs and Lowers ERG Protein Levels

This example demonstrates that the reduction of AR target gene expression result from a reduction of AR binding to AR gene target promoter sites.

To determine whether AR protein depletion from its target genes' androgen response elements (AREs) was responsible for reduced target gene expression, chromatin immunoprecipitation (ChIP) was performed. Cells were cross-linked with formaldehyde and reactions were stopped with glycine. Crosslinked cells were re-suspended in RIPA with SigmaFast protease inhibitors tablets (Sigma, St. Louis, Mo.) and sonicated on ice using a Branson Digital Sonifer model 450 (Branson Ultrasonics Co., Danbury, Conn.). Sonicated cellular lysates were used for Matrix ChIP (Flanagin et al., Nucleic Acids Res 36: e17, 2008) Immunoprecipitations were performed without adding an antibody or with 500 ng of an anti-AR antibody. PCR reactions were amplified using a thermocycler (see supplementary methods). ChIP PCR primer sequences were as follows:

PSA ARE ChIP Sense Primer:
(SEQ ID NO: 4)
GCCTGGATCTGAGAGAGATATCATC
PSA ARE ChIP Anti-Sense Primer:
(SEQ ID NO: 5)
ACACCTTTTTTTTTCTGGATTGTTG
TMPRSS2 ARE ChIP Sense Primer:
(SEQ ID NO: 6)
TGGTCCTGGATGATAAAAAAAGTTT
TMPRSS2 ARE ChIP Anti-Sense Primer:
(SEQ ID NO: 7)
GACATACGCCCCACAACAGA

Primers were synthesized by IDT (Coralville, Iowa). ChIP PCR program consisted of: 95° C. for 5 minutes for 1 cycle; 95° C. for 30 seconds, 58° C.(TMPRSS2-ERG) or 60° C. (PSA) for 30 seconds, and 72° C. for 30 seconds for 35-40 cycles; 72° C. final extension for 5 minutes. PCR products were run on 2% NaBO3 agarose gels containing GelStar dye (Cambrex, East Rutherford, N.J.) for visualization (Wang et al., Mol Cell 27: 380-39, 2007; Yoon & Wong, Mol Endocrinol 20: 1048-1060, 2006). Bands were imaged for densitometry using a Gel Doc XR UV transilluminator and Quantity One software (Bio-Rad, Hercules, Calif.).

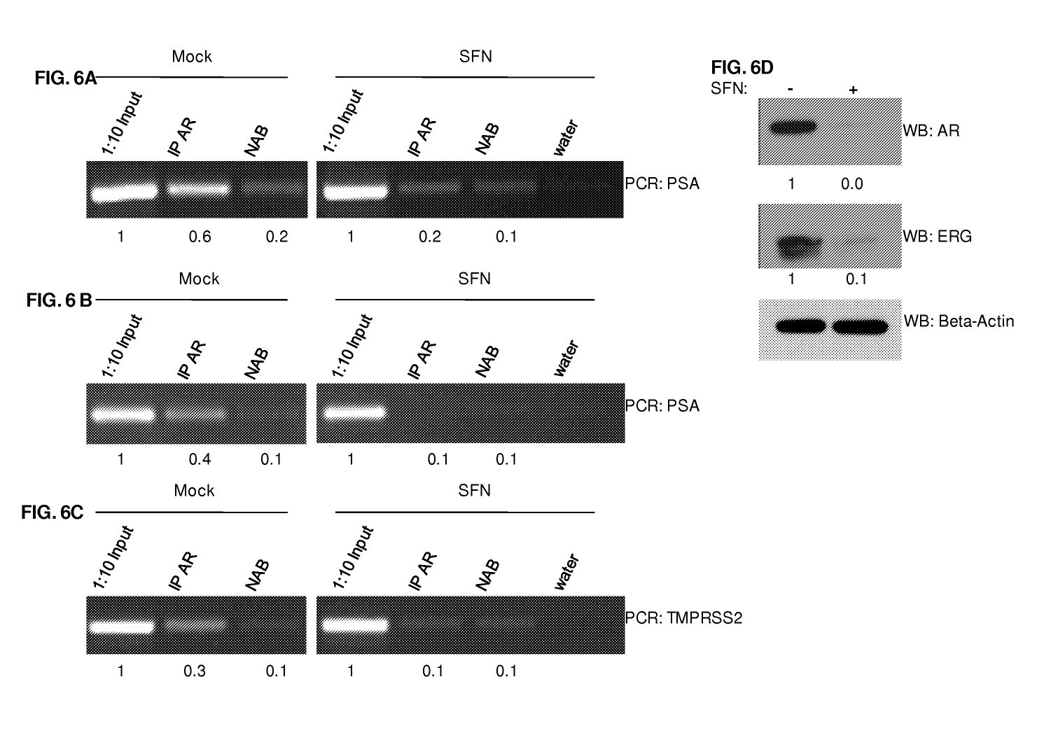

The results of the ChIP experiments showed that sulforaphane treatment lead to reduced enrichment of AR at the PSA ARE versus vehicle-treated cells (FIGS. 6A and 6B). This result matched the whole cell depletion that was observed for AR at this time point and dose of sulforaphane (FIGS. 2A and 2B). Similar findings were observed for VCaP cells at PSA and TMPRSS2 AREs (FIGS. 6B and 6C). A Western blot confirms near absent levels of AR and ERG protein at this same time point in whole cell lysates from VCaP cells treated in parallel with the cells processed for ChIP (FIG. 6D). This result indicates that reduced AR target gene occupancy parallels whole cell AR depletion and that reduced ERG mRNA parallels reduced ERG protein (FIGS. 5C, 6C, and 6D).

The disappearance of AR corresponded to a reduction in AR binding to and expression of its target genes. While it is possible that sulforaphane affects other AR co-factors, the reduced enrichment of AR at its target gene AREs by ChIP shows that AR depletion is mediating the reduction in AR target gene expression. Notably, levels of AR target genes including TMPRSS2-ERG, a fusion of the AR-regulated TMPRSS2 gene and the ERG oncogene which is commonly over-expressed in human cancer, are reduced. ERG over-expression in normal prostate cells leads to enhanced invasiveness and growth, and ERG knockdown in VCaP prostate cancer cells by siRNA leads to decreased invasiveness. Thus, therapies such as sulforaphane, which deplete cells of ERG protein, could be used for the prevention and treatment of prostate cancer. In addition, levels of PSA, whose serum protein levels are used to assess therapeutic efficacy in men with prostate cancer, are also lowered with sulforaphane.

### Example 5

### Proteasome Inhibitor Treatment Rescues AR Protein from Sulforaphane-Induced Degradation

Inhibition of HSP90 through increased acetylation leads to targeting of HSP90 client proteins to the proteasome for post-translational degradation. Because

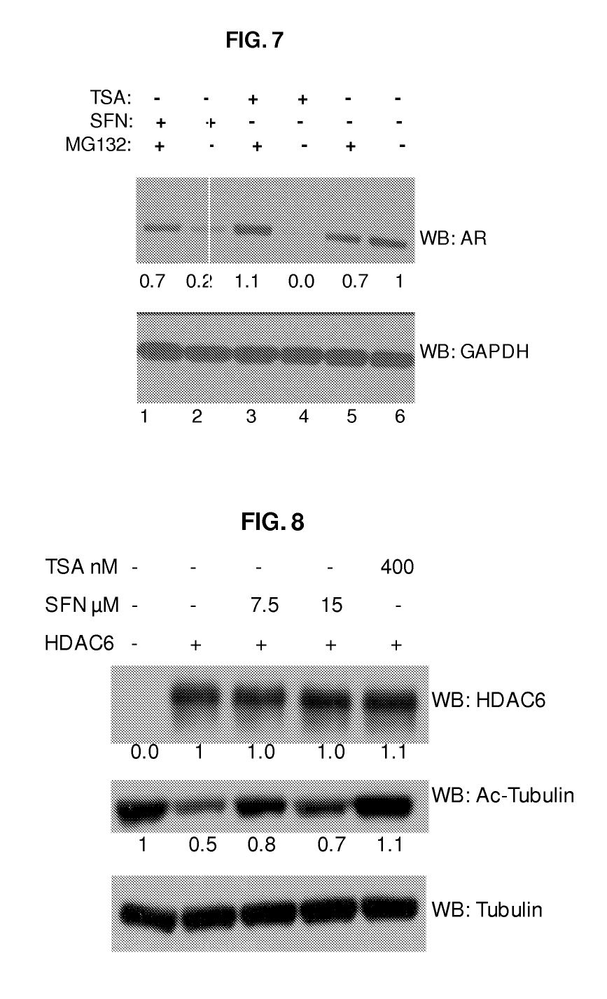

AR protein was observed as early as 6-12 hours after treatment with sulforaphane (see e.g., FIG. 2A), it was determined whether AR degradation could be rescued by inhibition of the proteosome degradation pathway. The effect of treatment with sulforaphane, Trichostatin A (TSA, #T8552 Sigma, St. Louis, Mo.), a pan-HDAC inhibitor, or vehicle was determined in the presence of the proteasome inhibitor MG132 (#PI-102-0005, Biomol, Plymouth Meeting, Pa.) or its vehicle (DMSO or ethanol) (FIG. 7). Treatment with sulforaphane (lane 2) or TSA (lane 4) in the absence of MG132 led to dramatic reductions in AR protein versus the vehicle-treated cells (lane 6). However, simultaneous treatment with MG132 and either sulforaphane (lane 1) or TSA (lane 3) restored the AR steady state level close to that seen in the vehicle-treated control (lane 5). Finally, treatment with MG132 alone did not change AR protein levels versus vehicle treatment (lanes 5 and 6). This highlights the fact that at 24 hours, the rescue effect with MG132 occurs only after perturbation to AR protein, which was observed with both sulforaphane and TSA treatments.

Sulforaphane treatment lead to increased acetylation of two HDAC6 target proteins: HSP90 and alpha-tubulin. In the setting of unchanged HDAC6 protein levels, HSP90 becomes hyperacetylated (seen as early as 4 hours after treatment), and the AR protein dissociates from HSP90. At later time points (as early as 6 hours), AR protein levels decline, an effect mediated by the proteasome (FIG. 7). It is possible that sulforaphane treatment marks AR protein for degradation through increased ubiquitylation as has been shown for certain client proteins which have dissociated from HSP90 after treatment with other HDAC inhibitors. The quick onset of AR protein decline (within 6-12 hours, FIG. 5) also indicated that post-translational mechanisms are at play. Given the results reported here with HSP90, sulforaphane may have therapeutic implications for other cancers due to the fact that HSP90 chaperones other key proteins including ErbB2 (protein product of the Her2Neu gene) in breast cancer, bcr-abl in CML, and c-kit in GIST.

### Example 6

### Sulforaphane Inhibits HDAC6 Enzymatic Function

The example describes methods used to show recombinant HDAC6 enzymatic activity on its tubulin substrate in a cell-free tubulin deacetylase assay. Two μg of recombinant HDAC6 (Biomol, Plymouth Meeting, Pa.) was used in a cell-free tubulin deacetylase assay with 25 μg of MAP-rich polymerized tubules (Cytoskeleton, Denver, Colo.) similar to Hubbert et al. (Nature 417: 455-458, 2002). Tubulin dimers were either incubated without recombinant HDAC6 or with recombinant HDAC6 in the presence of vehicle, sulforaphane, or TSA. Levels of HDAC6, acetylated tubulin, and tubulin by Western blot were quantified.

As shown in FIG. 8, incubation of vehicle-treated recombinant HDAC6 with tubulin dimers (lane 2) reduced levels of acetylated tubulin versus the vehicle-treated tubulin dimers incubated without HDAC6 (lane 1), which indicates that the HDAC6 enzyme was functional. However, incubation of HDAC6 with sulforaphane (lanes 3, 4) or TSA (lane 5) led to decreased HDAC6 deacetylase activity as evidenced by increased levels of alpha-tubulin compared with the vehicle-treated HDAC6 control (lane 2).

### Example 7

### HDAC6 Over-Expression Rescues AR and HDAC6 from Sulforaphane-Induced Degradation and Reverses Sulforaphane-Induced Acetylation of Alpha-Tubulin

This example describes methods used to demonstrate that HDAC6 over-expression rescues AR and HDAC6 proteins from sulforaphane treatment.

LNCaP cells were transiently transfected with pCDNA3.1 empty vector (Invitrogen, Carlsbad, Calif.) or FLAG-tagged HDAC6 expression vector (Addgene, Cambridge, Mass.) using Lipofectamine LTX and Plus reagents (Invitrogen, Carlsbad, Calif.). Forty-eight hours later, media was replaced for a 16-hour treatment with 15 nM sulforaphane or vehicle. Levels of FLAG, HDAC6, AR, acetylated alpha-tubulin, and actin were measured by Western blot.

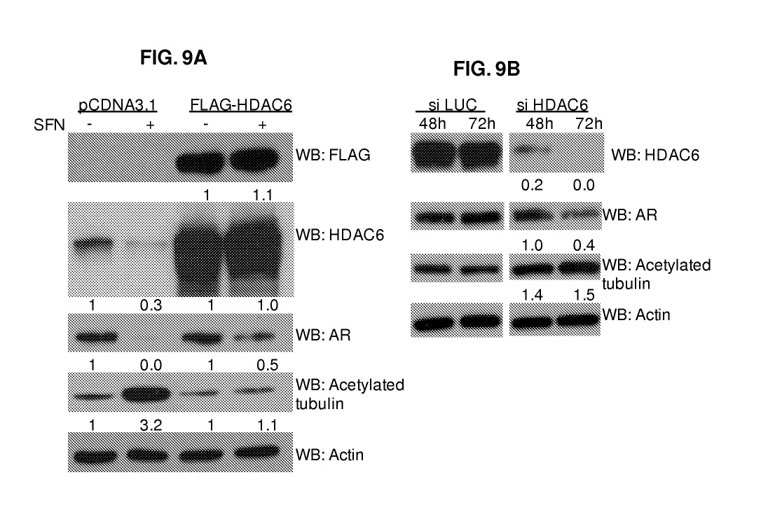

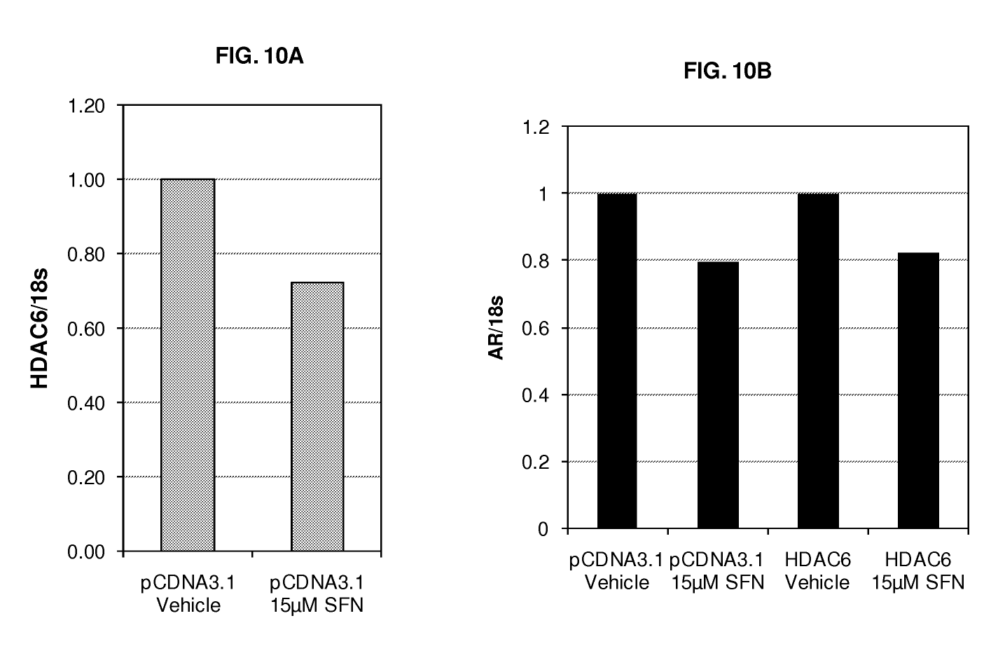

As shown in FIG. 9A, over-expression of HDAC6 led to detectable anti-FLAG signal and higher levels of HDAC6 versus empty-vector-transfected cells while levels of acetylated alpha-tubulin declined. This result indicated the presence of functional HDAC6 protein. In empty-vector transfected cells, AR protein is decreased and acetylated tubulin is increased with sulforaphane versus vehicle treatment. More interestingly, HDAC6 protein levels were also reduced with sulforaphane treatment under these conditions. While sulforaphane does reduce HDAC6 transcript levels, this does not fully account for the observed HDAC6 protein depletion (FIG. 10A).

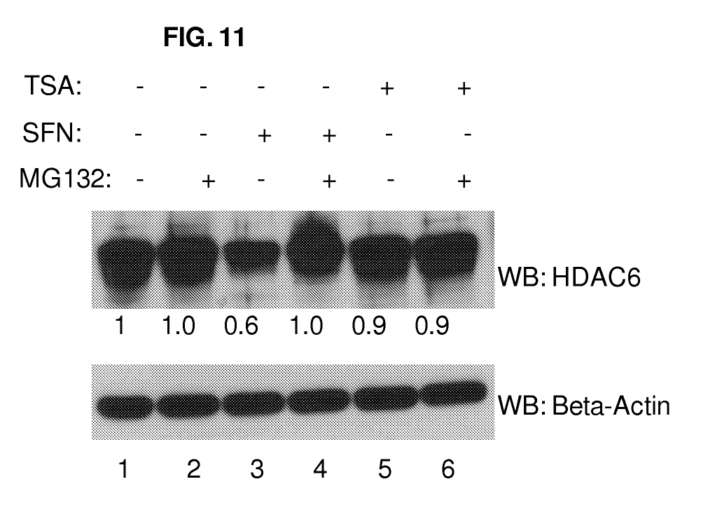

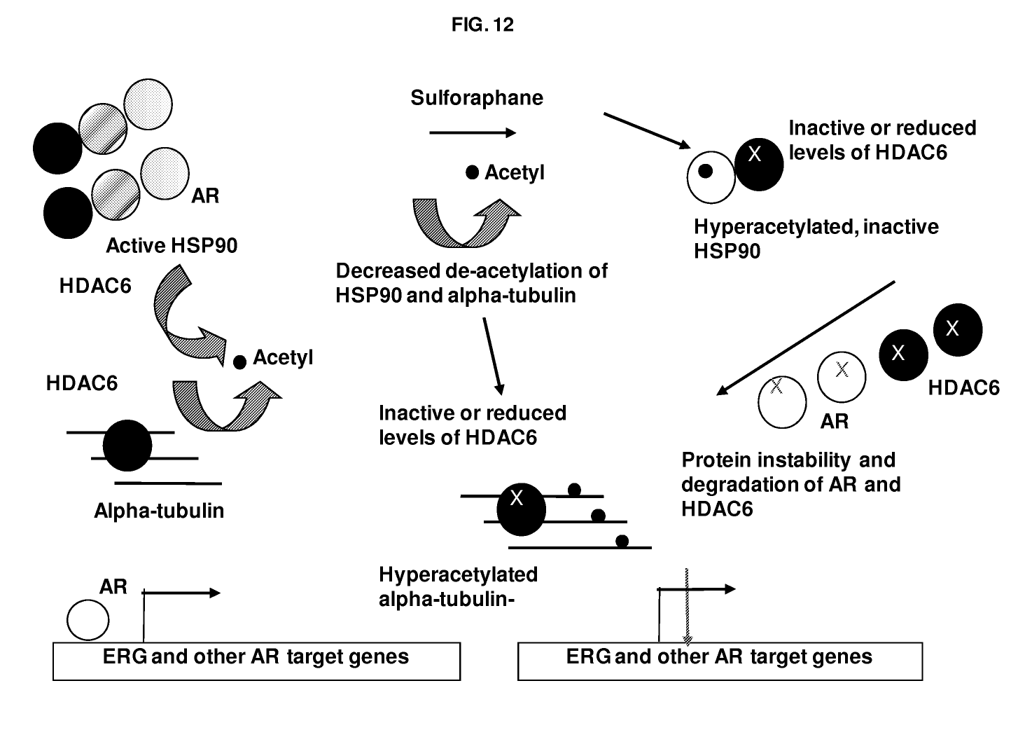

Concomitant proteasome inhibitor and sulforaphane treatment (lane 4) restored HDAC6 protein levels versus sulforaphane-treated cells (lane 3) while TSA had minimal effects on HDAC6 protein levels (lanes 5, 6) (FIG. 11). Additionally, in empty-vector transfected cells, sulforaphane treatment lowered AR protein and increased acetylation of alpha-tubulin. However, in HDAC6-over-expressing, sulforaphane-treated cells, HDAC6 protein expression was restored, AR protein depletion was attenuated (without changing AR transcript levels), and alpha-tubulin acetylation was blunted versus the effects seen in empty-vector, sulforaphane-treated cells (FIG. 9A, FIG. 10B).

### Example 8

### HDAC6 siRNA Recapitulates Sulforaphane's Effect on AR Protein Levels

LNCaP cells were transiently transfected with either a siRNA sequence complementary to the luciferase gene (CGTACGCGGAATACTTCGA; SEQ ID NO: 8) or to HDAC6 (CTGCAAGGGATGGATCTGAAC; SEQ ID NO: 9) using DharmaFECT 3 transfection reagent for a final concentration of 100 nM (Dharmacon, Lafayette, Colo.). At 48, 72, and 96 hours post-transfection, cells were harvested. Levels of protein expression by Western blot were quantified.

Compared with cells transfected with the luciferase siRNA, siRNA to HDAC6 led to depletion of HDAC6, which was most pronounced at 72 and 96 hours (FIG. 9B). At these same time points, AR protein was also diminished in the HDAC6 knockdown cells. Likewise, the effects of HDAC6 siRNA on alpha-tubulin acetylation were most pronounced at 72 and 96 hours, versus the luciferase siRNA cells, and recapitulated the effects seen with sulforaphane.

In summary, it is shown herein that sulforaphane treatment increases acetylation of two HDAC6 target proteins: HSP90 and alpha-tubulin. At early time points, in the setting of unchanged HDAC6 protein levels, HSP90 becomes hyperacetylated, and the AR protein dissociates from HSP90 (FIGS. 1A-C). At later time points, after treatment with sulforaphane or the HDAC inhibitor TSA, AR protein levels decline, and this decline is attenuated by inhibition of proteasomal degradation, which indicates that sulforaphane treatment targets AR for proteolytic degradation (FIGS. 2A-B and 7).

While for most doses of sulforaphane AR protein levels declined without reductions in AR transcript levels, for the highest doses of sulforaphane (20 μM in LNCaP cells and 15-20 μM in VCaP cells) AR transcript levels also declined. The HDAC inhibitor TSA also suppressed AR transcripts (FIGS. 4A-B). Consequently, the overall effect of sulforaphane and other agents with HDAC inhibitory properties on AR levels may not be solely post-transcriptional. It is clear, however, that a post-translational effect is a control point in light of the observations that AR protein levels are rescued by simultaneous sulforaphane and proteasome inhibitor treatment or over-expression of HDAC6 and that most sulforaphane doses do not lower AR transcript levels (FIGS. 4A-B, 7, 9A-B, 10A-B).

The disappearance of AR corresponded to reduced AR binding to and expression of its target genes (FIGS. 5A-C and 6A-D). The reduced enrichment of AR at its target genes by ChIP demonstrates that AR depletion is mediating the reduced AR target gene expression. Notably levels of TMPRSS2-ERG, a fusion of the AR-regulated TMPRSS2 gene and the ERG transcription factor, which is commonly over-expressed in human prostate cancer, were reduced. ERG over-expression in normal prostate cells leads to enhanced invasiveness and growth, and ERG knockdown in VCaP prostate cancer cells by siRNA leads to decreased invasiveness (Tomlins et al., Neoplasia 10: 177-188, 2008). ERG over-expression under an AR-regulated promoter in a transgenic murine model was recently shown by two independent groups to transform prostate cells and to induce formation of prostate cancer precursor lesions highlighting ERG's importance in early stages of prostate tumorigenesis (Tomlins et al., Neoplasia 10:177-188, 2008; Klezovitch et al., Proc Natl Acad Sci USA 105:2105-2110, 2008). Thus, therapies such as sulforaphane, which deplete cells of AR and ERG protein, may hold promise for the prevention and treatment of prostate cancer.

Incubation of recombinant HDAC6 with sulforaphane inhibited HDAC6 deacetylase activity in a cell-free system. In cells, sulforaphane treatment inhibits HDAC6 function without changing its levels at early time points (4 hours) (FIGS. 1A-C and 8). However, at later time points (16 hours), sulforaphane leads to reduced HDAC6 protein levels (FIGS. 9A and 11). The related compound CDDO-Imidazole also depletes HDAC6 in a dose-dependent manner, which parallels reduced AR protein levels and increased tubulin acetylation (FIGS. 3A and 3B). CDDO-Imidazole was previously shown to deplete Her2Neu protein levels in breast cancer cells through proteasomal degradation, although the effect of this agent on HDAC6 was not explored (Konopleva et al., Mol Cancer Ther 5:317-328, 2006). Given that CDDO-Imidazole, like sulforaphane, acts via the Nrf2 pathway, it is possible that the effect of these agents on HDAC6 protein levels is mediated by activation of gene targets of the Nrf2-Keap1 pathway.

There are several possible mechanisms for the reduced HDAC6 protein levels (beyond reduced HDAC6 transcript levels) (FIGS. 9A and 10A); however, the data with proteasome inhibitor rescue indicates that proteasomal degradation is principal (FIG. 11). The importance of reduced HDAC6 levels with sulforaphane treatment is highlighted by the fact that over-expression of HDAC6 protein, in the presence of sulforaphane, reverses sulforaphane's effect on depleting HDAC6 and AR proteins and increasing acetylation of alpha-tubulin compared with empty-vector transfected, sulforaphane-treated cells (FIG. 9A).

Finally, that HDAC6 depletion by siRNA recapitulates the sulforaphane treatment effects and leads to reduced AR protein further corroborates that the effects observed with sulforaphane are mediated by inactivation of HDAC6 (FIG. 9B). While it is possible that sulforaphane-mediated HSP90 hyperacetylation and consequent AR degradation occurs via inhibition of HSP90 deacetylases besides HDAC6, though none are known, the influence of this agent on HDAC6 function and protein expression accounts, at least in part, for the influence of sulforaphane on the expression of the AR protein (FIGS. 7, 9A and 9B).

Prostate cancer remains a common and sometimes lethal cancer, and it is clear that the AR protein is an important target in all phases of this disease. While the pre-clinical data and limited human data for sulforaphane is promising, the data provided herein elucidates new mechanisms of action of sulforaphane through non-histone protein deacetylase inhibition-increased protein acetylation of HDAC6 targets such as alpha-tubulin and HSP90, inhibition or reduced levels of HDAC6 protein, and consequently reduced levels of AR protein and target genes including PSA and TMPRSS2-ERG.

### Example 9

### Detection of HDAC6 Ablation in a Mouse Model of Prostate Cancer after Administration of Sulforaphane

This example describes efficacy teasting of a HDAC6 inhibitors in ablating HDAC6 levels in an a mouse model. Although particular methods, dosages, and modes of administrations are provided, one skilled in the art will appreciate that variations can be made without substantially affecting the treatment.

A mouse model has been developed in which ERG overexpresion, driven by AR, leads to prostate cancer precursor lesions. Klezovitch et al., PNAS 105(6):2105-10. Cohorts of mice are treated with Sulforaphane or vehicle control. The sulforaphane can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose. The agent can be administered in several doses, for example continuously, daily, weekly, or monthly. The mode of administration can be any used in the art. The amount of agent administered to the subject can be determined by a clinician, and may depend on the particular subject treated. Specific exemplary amounts are provided herein (but the disclosure is not limited to such doses).

The mice are euthanized and the prostates removed for subsequent examination. The extracted prostate tissue is examined for HDAC6 protein expression, and in some examples one or more of acetylated HSP-90, acetylated alpha-tubulin, AR, PSA, and TMPRSS2-ERG protein expression. Mice are also examined to determine efficacy of treatment, for example for tumor reduction. In some examples, the mice are treated one to four times daily with sulforaphane at a concentration of between 0.02 μg/gram body weight to 1.0 g/gram body weight for between one day and fifty days.

### Example 10

### Detection of HDAC6 Ablation in Patient Samples after HDAC6 Inhibitor Administration

This example describes efficacy teasting of a HDAC6 inhibitors in ablating HDAC6 levels in an samples obtained from patients. Although particular methods, dosages, and modes of administrations are provided, one skilled in the art will appreciate that variations can be made without substantially affecting the treatment.

Subjects are selected that have or are suspected of having prostate cancer, for example because of high levels of prostate specific antigen (PSA). Briefly, the method can include screening subjects to determine if they have prostate cancer. Subjects having prostate cancer are selected. In some examples, prostate cancer testing consists of initial screening to detect elevated levels of PSA, such as with an enzyme-linked immunosorbent assay (ELISA) to detect increased PSA.

Following subject selection, a therapeutic effective dose of an HDAC6 inhibitor, such as sulforaphane, is administered to the subject. The HDAC6 inhibitor can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose. In one example, sulforaphane is administered orally to a human subject having prostate cancer at a dose of 50 to 1000 μg/day, such as 200 to 400 μg/day. In another example, tubacin is administered orally to a human subject having prostate cancer at a dose of 50 to 1000 μg/day, such as 200 to 400 μg/day. In yet another example, belinostat is administered i.v. over 30 minutes to a human subject having prostate cancer at a dose of 50 to 2000 mg/m2/day, such as 50 to 1000 mg/m2/day on days to 5 of a 21 day cycle. However, the particular dose can be determined by a skilled clinician. The agent can be administered in several doses, for example continuously, daily, weekly, or monthly. The mode of administration can be any used in the art. The amount of agent administered to the subject can be determined by a clinician, and may depend on the particular subject treated. Specific exemplary amounts are provided herein (but the disclosure is not limited to such doses).

The amount of the composition administered to prevent, reduce, inhibit, and/or treat prostate cancer or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (for example, prostate cancer) in a subject without causing a substantial cytotoxic effect in the subject. An effective amount can be readily determined by one skilled in the art, for example using routine trials establishing dose response curves. The therapeutic compositions can be administered in a single dose delivery, via continuous delivery over an extended time period, in a repeated administration protocol (for example, by a daily, weekly, or monthly repeated administration protocol). Therapeutic compositions can be taken long term (for example over a period of months or years).

Prostate tissue samples are obtained from subjects, such as subject prior to treatment with an HDAC6 inhibitor, after HDAC6 inhibitor treatment, and/or in recurrent cancers in which the levels of PSA has increased. The prostate tissue is examined for HDAC6 protein expression, and in some examples one or more of acetylated alpha-tubulin, acetylated HSP90, AR, PSA, or TMPRSS2-ERG protein expression. In some examples the tissue samples are subjected to immunoassays, such as ELISA assays for HDAC6 to determine if the subject is responsive to HDAC6 inhibitor treatment, such as sulforaphane treatment.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method for identifying a mammalian subject as a candidate for treatment with an inhibitor of HDAC6 deacetylase activity, comprising:
contacting a biological sample obtained from the subject with an inhibitor of HDAC6 deacetylase activity;
detecting an amount of HDAC6 protein in the biological sample; and
comparing the amount of HDAC6 protein in the biological sample with a control, wherein a reduction in the amount of HDAC6 protein in the biological sample relative to the control indicates that the subject is a candidate for treatment with an inhibitor of HDAC6 deacetylase activity.

2. A method of monitoring response to a cancer treatment, comprising:
detecting an amount of HDAC6 protein present in a biological sample obtained from a mammalian subject; and
comparing the amount of HDAC6 protein with a control, wherein a decrease in the amount of HDAC6 protein relative to a control indicates that the subject is responding to the cancer treatment, and an increase or maintenance in the amount of HDAC6 protein relative to the control indicates that the subject is not responding to the cancer treatment.

3. A method for selecting a compound for the treatment of cancer in a subject, comprising:
contacting a biological sample obtained from a subject with a test agent;
detecting an amount of HDAC6 protein in the biological sample; and
comparing the amount of HDAC6 protein detected to a control, wherein a reduction in the amount of HDAC6 protein relative to the control indicates that the agent is of use in treating the cancer in the subject.

4. The method of claim 1, wherein the biological sample is prostate tumor sample.

5. The method of claim 1, wherein the control comprises the amount of HDAC6 protein in a normal prostate sample.

6. The method of claim 4, wherein the control sample comprises a biological sample not contacted with an inhibitor of HDAC6 deacetylase activity, a sample of non-cancerous cells, or cells obtained from the subject from an earlier time point.

7. (canceled)

8. (canceled)

9. The method of claim 1, wherein the subject is a human.

10. The method of claim 1, wherein the inhibitor of HDAC6 deacetylase activity is sulforaphane.

11. The method of claim 1, wherein the subject has a tumor and a decrease in the amount of HDAC6 protein in the biological sample relative to the control indicates that the tumor in the subject is sensitive to treatment with the inhibitor of HDAC6 deacetylase activity.

12. The method of claim 10, wherein the tumor is a prostate tumor.

13. The method of claim 2, wherein the cancer is prostate cancer.

14. The method of claim 3, wherein the subject has a prostate tumor.

15. (canceled)

16. The method of claim 1, further comprising:
detecting an amount of one or more of acetyl HSP90 protein, acetyl alpha-tubulin protein, androgen receptor (AR) protein, prostate specific antigen (PSA) protein, or TMPRSS2-ERG in the biological sample; and
comparing the amount of the acetyl HSP90 protein, acetyl alpha-tubulin protein, AR protein, PSA protein, or TMPRSS2-ERG protein in the biological sample with a control, wherein a reduction in the amount of AR protein, PSA protein, or TMPRSS2-ERG protein or an increase in the amount of acetyl HSP90 protein, acetyl alpha-tubulin protein in the biological sample relative to the control indicates (a) that the subject is a candidate for treatment with an inhibitor of HDAC6 deacetylase activity, (b) that the subject is responding to the cancer treatment, or (c) that the test agent is of use in treating the cancer in the subject.

17. A method for identifying an agent that inhibits cancer, comprising:
contacting at least one isolated cell with one or more test agents;
detecting an amount of HDAC6 protein in the cell; and
comparing the amount of HDAC6 protein detected to a control, wherein a reduction in the amount of HDAC6 protein relative to the control indicates that the one or more test agents inhibit cancer.

18. The method of claim 17, further comprising:
detecting an amount of one or more of acetyl HSP90 protein, acetyl alpha-tubulin protein, androgen receptor (AR) protein, prostate specific antigen (PSA) protein, or TMPRSS2-ERG in the biological sample; and
comparing the amount of the acetyl HSP90 protein, acetyl alpha-tubulin protein, AR protein, PSA protein, or TMPRSS2-ERG protein in the biological sample with a control, wherein a reduction in the amount of AR protein, PSA protein, or TMPRSS2-ERG protein or an increase in the amount of acetyl HSP90 protein, acetyl alpha-tubulin protein in the biological sample relative to the control indicates that the one or more test agents inhibit cancer

19. (canceled)

20. The method of claim 17, wherein the cell is a LNCaP cell or a VCaP cell.

21. The method of claim 17, wherein the cancer is prostate cancer.

22. (canceled)

23. The method of claim 17, wherein detecting the amount of the protein comprises one or more of an immunohisto chemical assay, a radioimmunoassay, a Western blot assay, an immunofluorescent assay, an enzyme immunoassasy, a chemiluminescent assay, or a mass spectrometry assay.

24. (canceled)

25. (canceled)

26. (canceled)

27. A kit, comprising:
an HDAC6-specific antibody; and
one or more of an androgen receptor-specific antibody, prostate specific antigen-specific antibody, or TMPRSS2-ERG-specific antibody.

28. The kit of claim 27, further comprising an anti-acetyl alpha-tubulin antibody.
